(19) United States
(12) Patent Application Publication
(10) Pub. No.: US 2026/0000544 A1
(43) Pub. Date: Jan. 1, 2026

(54) DURABLE ABSORBENT UNDERWEAR WITH ODOR CONTROL FEATURES

(71) Applicant: The Procter & Gamble Company, Cincinnati, OH (US)

(72) Inventors: Matthew F. COPELAND, Mason, OH (US); Ian Murray Cameron FLEMING, Morrow, OH (US); Mackenzie Jean CHILDS, Cincinnati, OH (US); Jennifer Nichole PYLE, Madeira, OH (US)

(21) Appl. No.: 19/254,092

(22) Filed: Jun. 30, 2025

Related U.S. Application Data
(60) Provisional application No. 63/665,298, filed on Jun. 28, 2024.

(51) Int. Cl. A61F 13/00; A61F 13/15; A61F 13/51; A61F 13/538

(52) U.S. Cl. CPC A61F 13/00063; A61F 13/538; A61F 2013/15544; A61F 2013/5109

(57) ABSTRACT

A durable absorbent pant may include an absorbent gusset disposed in at least a crotch portion, the absorbent gusset comprising at least one layer of absorbent fabric and a fluid barrier layer disposed beneath the at least one layer of absorbent fabric; the absorbent fabric having a forwardmost end edge in the front portion, a rearwardmost end edge in a rear portion, and a pair of opposing side edges proximate the respective leg opening edges; a peripheral region; and an odor-adsorbent structure disposed at least partially in a peripheral region, the odor-adsorbent structure comprising an adsorbent layer comprising an adsorbent material selected from the group consisting of zeolites, functionalized silicas metal-organic frameworks, activated carbon, and combinations thereof.

### CROSS REFERENCE TO RELATED APPLICATION

This application claims the benefit of U.S. Provisional Application No. 63/665,298, filed Jun. 28, 2024, the substance of which is incorporated herein by reference.

### BACKGROUND

Many women have utilized conventional tampons and/or absorbent feminine hygiene pads to intercept, contain and absorb menstrual discharge, to avoid soiling outer clothing, bedclothes, etc. during menstruation. Relatively recently, however, offerings of durable/washable absorbent underwear products adapted for this purpose have begun to proliferate in the market. Such products may be used by some as a supplement to tampons and/or pads, and by others as a complete alternative. Market demand for such products may be increasing, for reasons that may include concerns with sustainability; advancements in suitable garment material compositions and underwear designs and manufacturing processes; greater convenience of use; efficacy; and other personal/individual choices.

When menstrual fluid is present within an absorbent structure following discharge, microorganisms that were present within the normal microbiome of the vagina and/or are present about the external surfaces of the skin and the proximate environment, may begin acting upon components of the fluid. Components of menstrual fluid, together with temperatures proximate the outer body surfaces, can support metabolism, growth, and reproduction of certain microbial species. As they act upon components of the fluid, some microbial species generate gases or volatile organic compounds that may become airborne and may be perceived as unpleasant odor(s). It is believed that a substantial number of women have concerns about odor during menstruation.

To prevent or reduce such odor, various materials have been identified and associated with products adapted to manage menstrual discharge. For durable/washable garments, such as durable absorbent underwear, challenges lie in incorporation of material that will withstand laundering and remain within the garment; will remain effective following a satisfactory number of launderings; is not irritating or harmful to the user's skin or vaginal areas, and is not disruptive to the normal (healthy) microbiome. Attempting to provide an odor control function while addressing these challenges, some manufacturers have incorporated antimicrobial components into underwear fabrics or other components. Many of such materials may include metallic elements such as copper, silver or zinc, or compounds thereof other materials that may be incorporated for antimicrobial effects may include organic acids or inorganic compounds. Such materials may have a lethal or growth-inhibiting effect on microbes, upon contact or proximity.

Inclusion of such antimicrobial materials, however, has encountered resistance in some market segments. Some consumers may believe that such materials may be directly or indirectly harmful to the wearer/user of the garment, or may contribute to development of bacterial strains that are resistant to the effects of such materials and/or are more robust. Additionally, use of some of such materials has fallen into disfavor by some regulatory authorities, for various reasons.

Accordingly, additional opportunity exists for development of absorbent underwear having features adapted to manage unpleasant odors that may result from microbial growth within discharged menstrual fluid, retained within an absorbent structure in durable absorbent underwear.

### BRIEF DESCRIPTION OF THE DRAWINGS

FIG. 1 is a simplified depiction of an example of a brief pant, as it would appear laid out flat on a horizontal planar surface, front waist portion facing up.

FIG. 2 is a depiction of an example of the brief pant of FIG. 1, in an assembled but opened configuration wherein the front and rear waist portions have been separated at the hip portions or hip side seams, as it would appear laid out flat on a horizontal planar surface, wearer-facing surfaces facing up.

FIGS. 3A-3C are partial, schematic lateral cross section views of examples of absorbent gusset assemblies, through a side edge proximate to a leg opening.

FIGS. 4A and 4B are partial, schematic longitudinal cross section views of examples of pants including absorbent gusset assemblies, through a crotch portion and leg opening edge.

FIGS. 5, 6A-6C, 7A-7C, 8 and 9 are partial, schematic longitudinal cross section views of additional examples of pants including absorbent gusset assemblies, through a crotch portion and leg opening edge.

FIGS. 10A-10C are partial, schematic longitudinal cross section views of examples of pants including absorbent gusset assemblies, through an end of the gusset assembly.

FIG. 11 is a schematic vertical cross section, through a longitudinal center plane (as identified in FIG. 1) of one possible example of a pant with a gusset, oriented as it would be oriented on a standing wearer.

FIG. 12A is a simplified depiction of an example of a brief pant, as it would appear laid out flat on a horizontal planar surface, front waist portion facing up.

FIG. 12B is a simplified depiction of an example of a shorts pant (or legged pant), as it would appear laid out flat on a horizontal planar surface, front waist portion facing up.

FIG. 13A is a plan view of an example of a gusset laid out on a horizontal surface, wearer-facing surface facing the viewer.

FIG. 13B is a schematic vertical cross section of a portion of the gusset shown in FIG. 13A.

FIG. 14A is a schematic depiction of an example of a brief pant, in an assembled but opened configuration wherein the front and rear waist portions have been separated at the hip portions or hip side seams, as it would appear laid out flat on a horizontal planar surface, wearer-facing surfaces facing up; FIG. 14A illustrates location of a “peripheral region” of a pant as the term is used herein.

FIGS. 14B-14F are schematic depictions of the example of the brief pant shown in FIG. 14A, further depicting examples of configurations of placements of odor adsorbent structures.

FIGS. 15A-15D are schematic partial cross sections taken along the z-direction, of examples of configurations of odor adsorbent structures.

### DETAILED DESCRIPTION

### Definitions

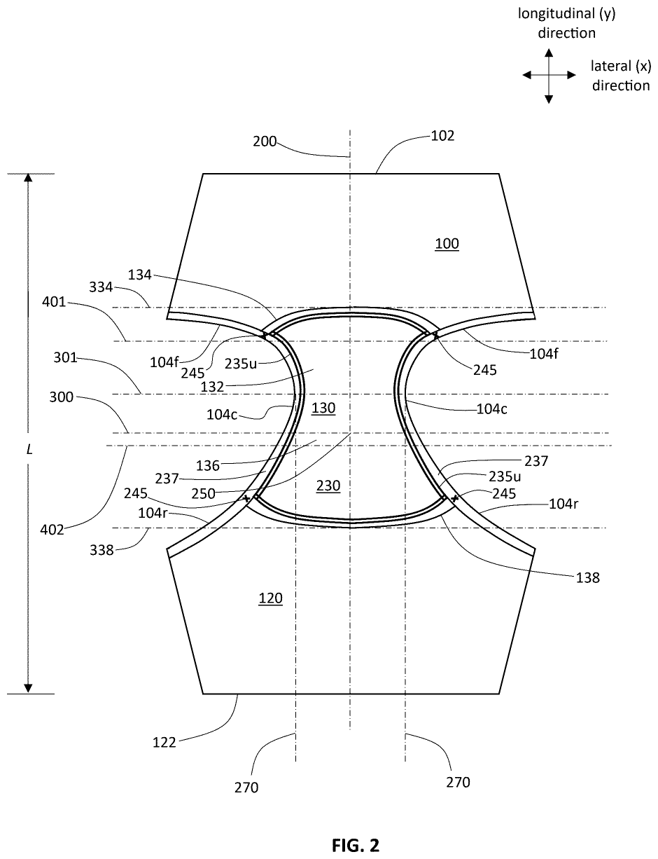

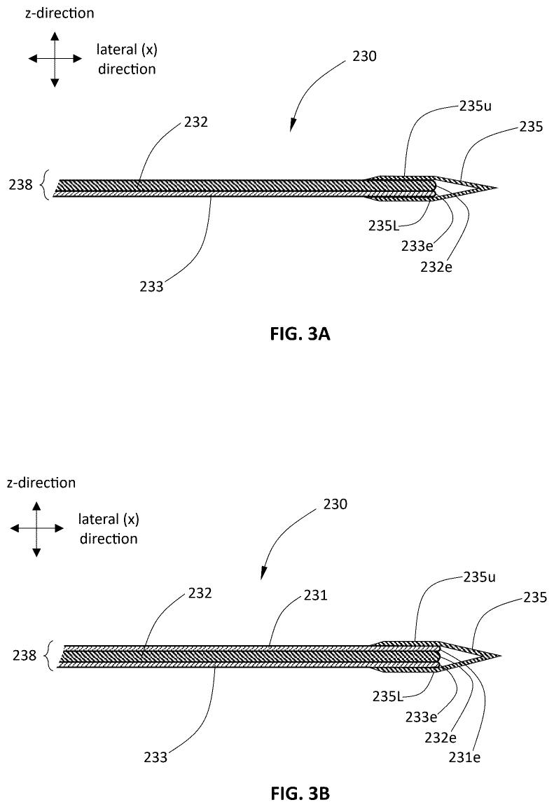

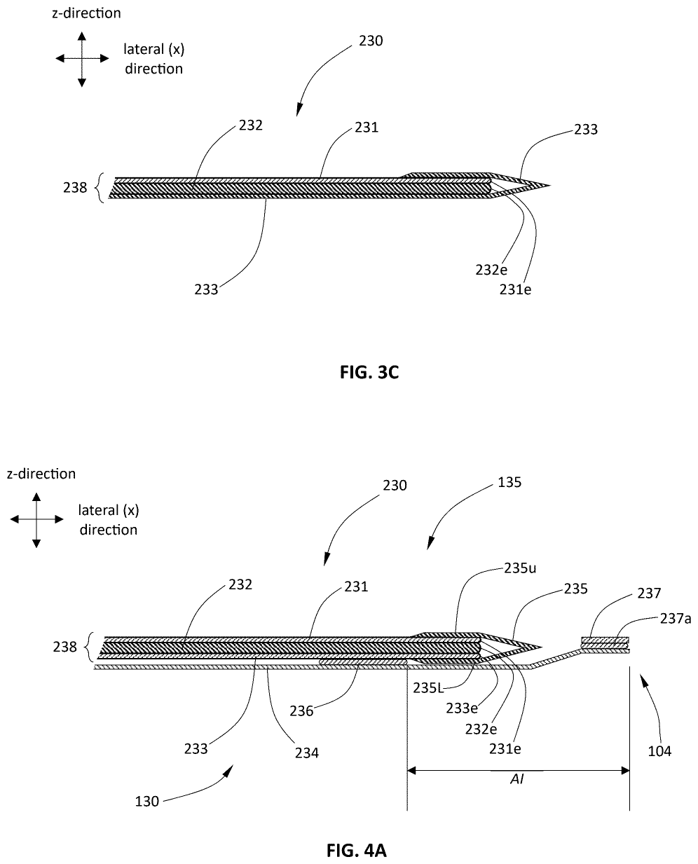

With respect to respective layer components in a gusset of a pant in an assembled but opened configuration (e.g., as illustrated in FIG. 2), laid out flat on a horizontal planar surface, wearer-facing surfaces facing up, as between first and second layer components in the crotch portion, the terms “above,” “upper,” “superadjacent,” “below,” “lower,” “subjacent” and/or “beneath” describe the components' disposition along the z-direction relative each other. Thus, for example, referring to FIG. 3B, a wearer-facing layer 231 is disposed “above” an absorbent layer 232 and a barrier layer 233, and conversely, the barrier layer 233 is disposed “below” the wearer-facing layer 231 the absorbent layer 232. “Superadjacent” and “subjacent” with respect to two layer components, mean, further, that the two layer components are disposed in direct surface-to-surface facing relationship (contact or uninterrupted proximity) with each other.

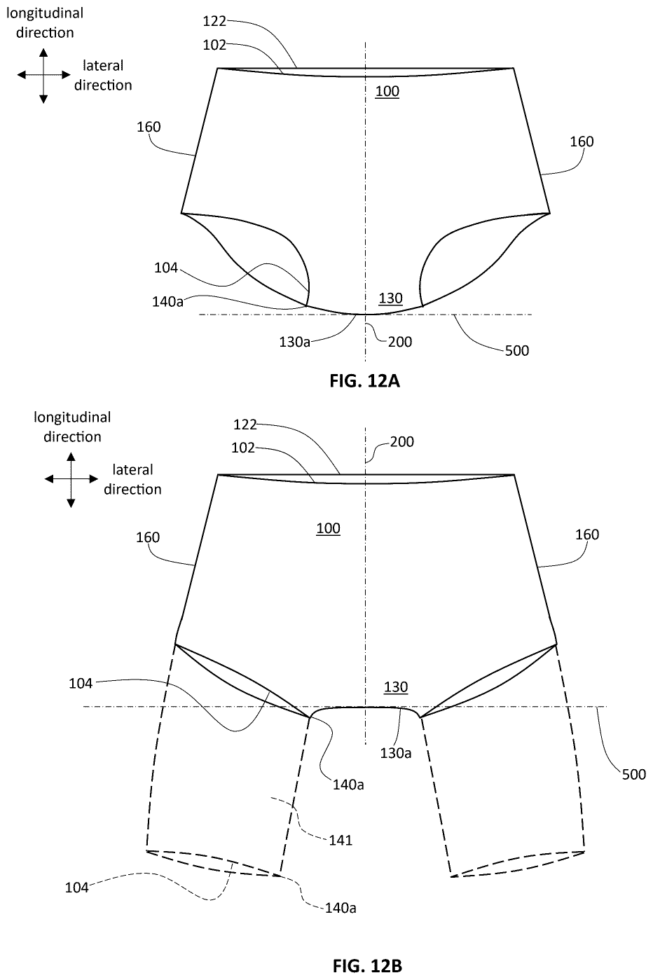

For purposes herein, a “brief pant” is distinguished from a “legged pant” by the configuration of the inside leg edges, resulting from the manner in which the component materials are shaped, sized, proportioned and seamed or otherwise affixed together. FIG. 12A depicts an example of a brief pant and FIG. 12B depicts an example of a legged pant in alternative forms. When the garment in its assembled condition is laid out flat on a horizontal planar surface, front waist portion facing up, and a lateral crotch tangent line 500 is drawn perpendicularly to longitudinal axis 200 and tangent to the point at which the crotch portion lower profile 130a intersects the longitudinal axis 200,
for a brief pant, the lowermost points 140a along the inside leg opening edges 104 are disposed along or above the crotch tangent line 500 (i.e., toward the front waist edge 102) (see FIG. 12A); and
for a legged pant, the lowermost points 140a along the inside leg opening edges 104 are disposed below the crotch tangent line 500 (i.e., away from the front waist edge 102) (see FIG. 12B).
   As reflected in FIG. 12B, for purposes herein, a legged pant may have leg portions 141 of any length, wherein the lowermost points 140a of inside leg opening edges 104 are disposed below the crotch tangent line 500. Underwear shorts pants such as “boy short” styles will generally have shorter leg portions as suggested in the solid-line portions of FIG. 12B, while other legged pants may have leg portions of varying lengths, as suggested by the dashed-line portions of FIG. 12B.

With respect to a wearable garment such as an underwear pant, “durable” means that the garment is made predominantly of fabric, and may be laundered or hand-washed, and dried, for reuse/re-wear a plurality of times without substantial loss of original shape, structural integrity, absorbent function or other useful mechanical attributes that existed prior to laundering or washing.

A yarn, thread, fiber, filament, web, film or fabric material, or a laminate or composite of any of these, is considered to be “elastically extensible” for purposes herein if, when a tensile force no greater than 50 gf/mm (gf per mm of sample width, where sample width is measured perpendicular to the stretch direction) is applied to the subject material along a stretch direction, the material may be extended along the direction to an elongated dimension of at least 130% of its original relaxed dimension (i.e., can extend at least 30%), without rupture or breakage which substantially damages the subject material; and when the force is removed from the subject material, the material retracts along the stretch direction to recover at least 40% of such elongation. To illustrate, if a section of fabric having an original relaxed length of 100 mm and a sample width of 40 mm can be elongated by tensile force of 2000 gf (50 gf/mm) in a direction along its length to 130 mm length without substantial damage, and will retract upon removal of the force to a length no greater than 118 mm (130 mm-118 mm=12 mm=40% of 30 mm), it is “elastically extensible” as defined herein. “Elongation,” used herein to quantify and express an amount of strain imparted to an elastically extensible material in a stretch direction, means: {[(strained length of the strand)−(length of the strand prior to straining)]/(length of the strand prior to straining)}, ×100%. For a monolithic material such as a cast or extruded film material, “elastomeric” means “elastically extensible” as defined herein.

As used herein, “fabric” means a cloth material that is knitted or woven of fibers, or threads or yarns of fibers. Herein, the term “fabric” does not include a film or a fibrous nonwoven web material, that latter of which is commonly used to form components of disposable diapers and disposable absorbent underwear.

As used herein, “film” means a skin- or membrane-like material that is cast, extruded or formed in place, from a molten thermoplastic material. Herein, the term “film” does not include fibrous nonwoven web material or fabric.

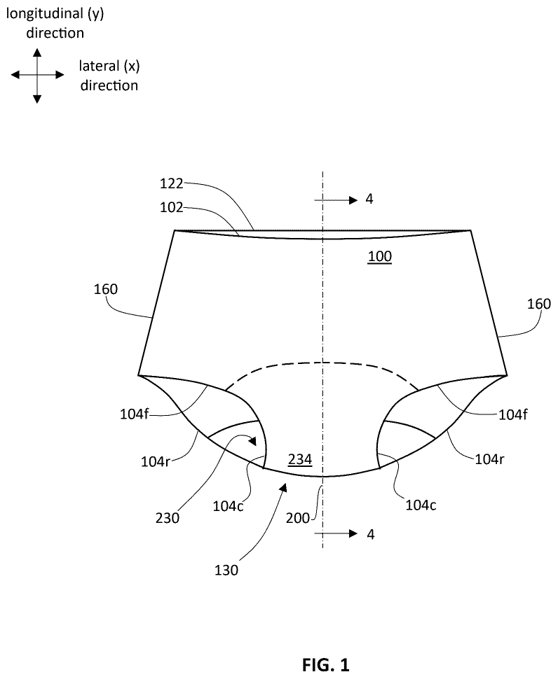

With respect to a pant in an assembled but opened configuration (e.g., as illustrated in FIG. 2), laid out flat on a horizontal planar surface, “lateral” refers to a direction generally parallel to a line tangent each of the left and right leg opening edges where they are closest the front waist edge. With respect to a pant in an assembled configuration (e.g., as illustrated in FIG. 1), laid out flat on a horizontal planar surface, front waist portion facing up, “lateral” refers to a direction generally parallel to a line tangent each of the left and right leg opening edges where they are closest the front waist edge. “Width” refers to a dimension measured along the lateral direction. The lateral direction may also be referred to herein as the “x-direction”.

With respect to a pant in an assembled but opened configuration, laid out flat on a horizontal planar surface, “longitudinal” refers to a direction generally perpendicular to a line tangent each of the left and right leg opening edges where they are closest the front waist edge. With respect to a pant in an assembled configuration, laid out flat on a horizontal planar surface, front waist portion facing up, “longitudinal” refers to a direction generally perpendicular to a line tangent each of the left and right leg opening edges where they are closest the front waist edge. “Length” refers to a dimension measured along the longitudinal direction. The longitudinal direction may also be referred to herein as the “y-direction”.

For purposes herein, “pant” includes any garment adapted for wear about the human lower torso, including a front waist portion and a rear waist portion that join about the wearer's hips and beneath the wearer's crotch, to form a garment having a waist opening and a pair of leg openings. Herein, the term “pant” encompasses (but is not limited to) a garment defined herein as a “brief pant”; a garment defined herein as a “legged pant”, and any other garment whether adapted for use as underwear or outerwear, having such features.

For purposes herein, unless otherwise specified, with respect to the proportionate content of a component material in a combination or structure, “predominant” means the component constitutes the majority of the weight of the combination or structure.

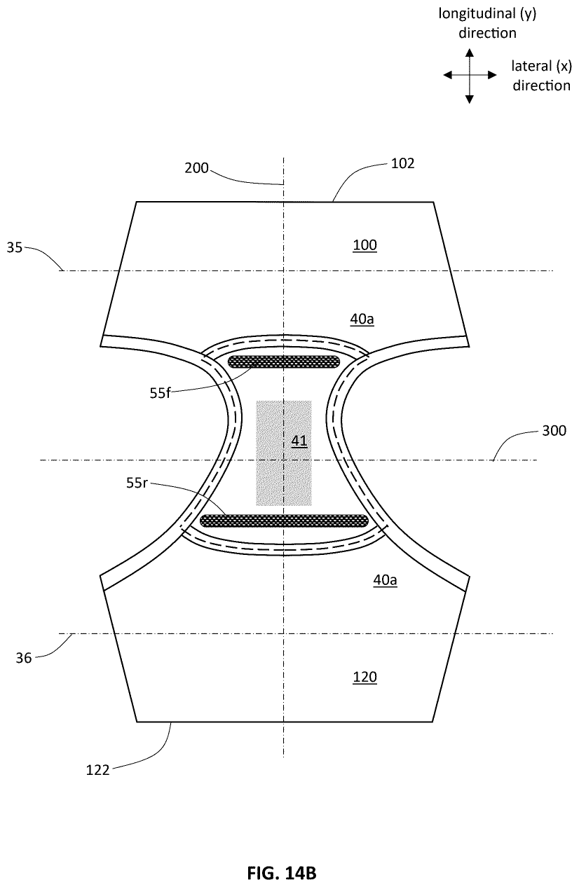

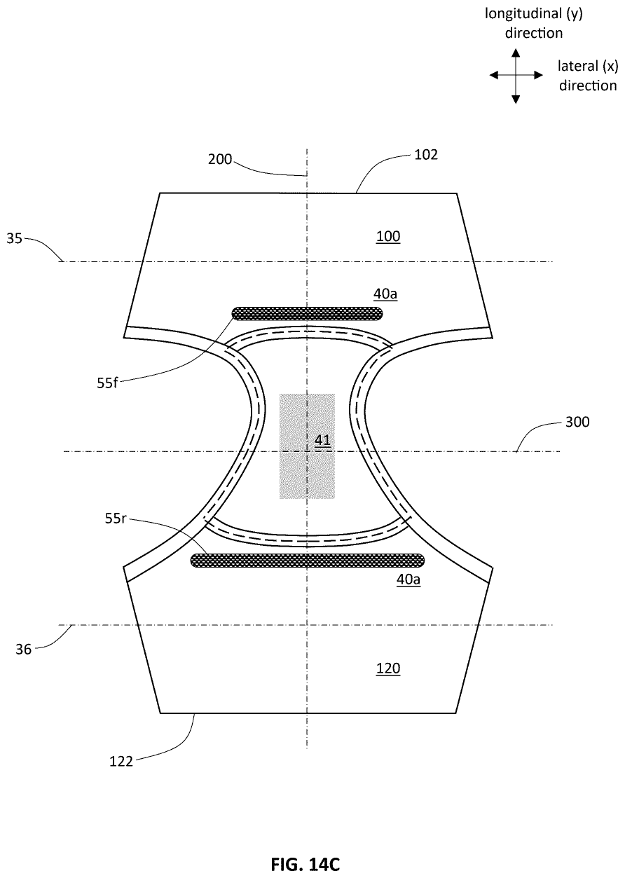

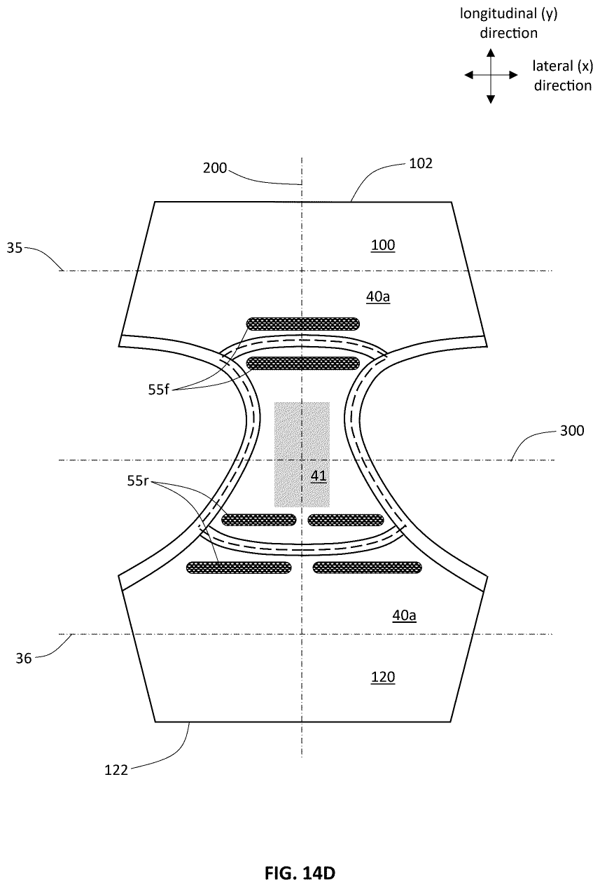

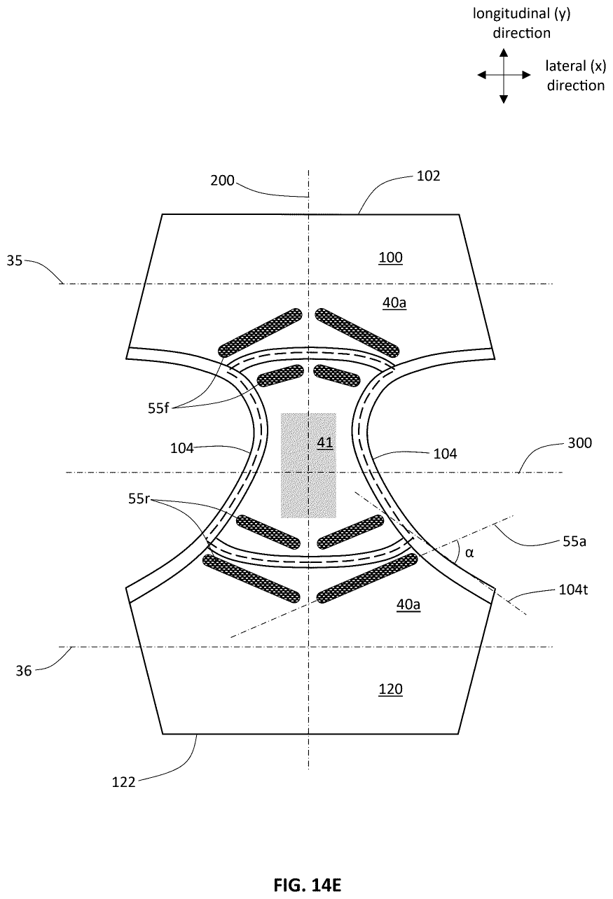

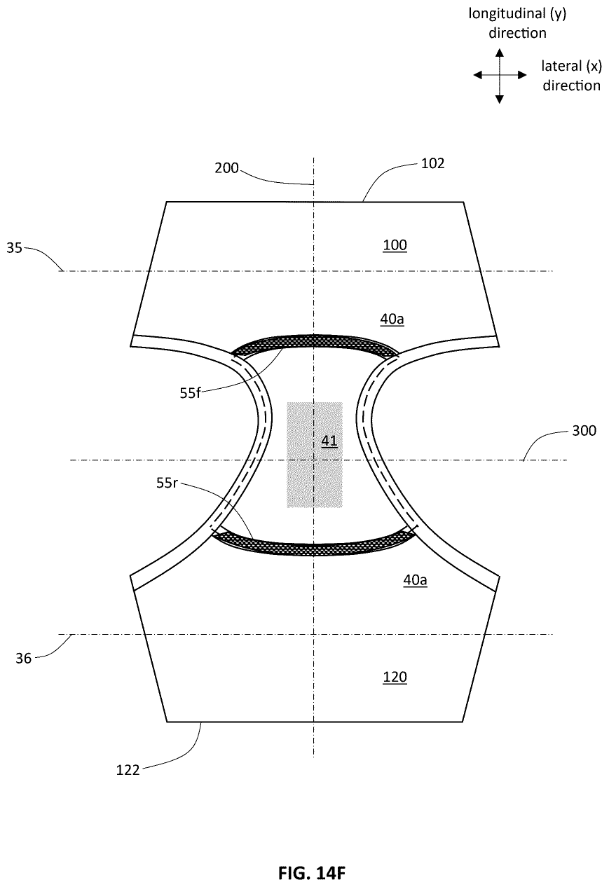

The “peripheral region” of an absorbent pant is the region laterally and longitudinally outside of the “central region” of the pant, as viewed in plan view in a configuration in which the pant has been separated at the side seams or hip regions, opened and laid out flat on a horizontal surface, wearer-facing surfaces facing upward. Referring to FIG. 14A, more particularly, the central region 41 (indicated by shading) is the region bounded at the outside front and rear thereof, by respective forward and rearward lateral lines 25, 26 that each equally divide the distance between the lateral axis 300 and the respective forwardmost and rearwardmost end edges 15, 16 of the absorbent fabric layer(s) of the absorbent gusset. The peripheral region 40 is the region outside the central region, as shown. The central region 41 is bounded at outside side extents thereof by respective inside lateral lines 461, 46r that respectively divide the distance between the respective proximate leg opening edges 104 at their laterally inwardmost extents, and longitudinal axis 200.

With respect to two oppositely-facing surfaces of a layer component of a pant, “wearer-facing” refers to the surface that faces the wearer's skin when the pant is worn normally; and “outward-facing” refers to the surface that faces away from the wearer's skin. With respect to two distinct layer components of a pant that are disposed respectively over and beneath each other, the “wearer-facing” layer component is the component that is disposed closest the wearer's skin when the pant is worn normally; and the “outward-facing” layer component is the component that is disposed farthest from the wearer's skin.

With respect to a pant in an assembled but opened configuration (e.g., as illustrated in FIG. 2), laid out flat on a horizontal planar surface, the “z-direction” is the direction orthogonal to the longitudinal and lateral directions, i.e., the vertical direction relative the horizontal planar surface.

### DESCRIPTION

One problem presented in designing an absorbent system to be used to contain and absorb discharged menstrual fluid, vaginal fluid, and unintended (incontinent) small discharges of urine by a woman results from adhering flow. As a result of relatively low velocity of discharges, female anatomical features, and typical surface chemistry of human skin (which can cause skin surfaces to attract and be relatively wettable by aqueous solutions such as menstrual fluid and urine), adhering flow has always been a phenomenon associated with menstruation, vaginal discharge, and low to moderate adult female incontinence. If features of a chosen containment/absorbency system do not reliably hold absorbent materials against the wearer's skin to intercept adhering flow, leakage can result.

It is believed, however, that a durable adult absorbent brief pant (i.e., a pant with a crotch portion including an absorbent structure and a liquid impermeable barrier layer to the outside thereof), that reliably protects against leakage of adhering flow through a variety of body movements and positions and over a reasonable duration of wear/use has not been marketed to date. Currently marketed pant products do not effectively hold included absorbent structures against the body through various body movements, over a reasonable duration of wear/use. It is believed that the garment industry to date has failed to recognize that suitably oriented elastic stretch is important for maintaining proximity and/or contact of the absorbent structure with the wearer's body contours and skin surfaces through various body positions and movements, and that a structure having a combination of absorbent capacity suitable for providing protection against leakage resulting from menstrual flow, vaginal discharge, or light to moderate incontinence, while having suitable directional elastic stretch so as to maintain effective contact of the structure with the wearer's body, may be designed. The human body and particularly the female lower torso and crotch region have geometrically non-ruled contoured surfaces. Fabrics or web materials that do not exhibit an effective amount of elastic stretch capability along an effective direction will not effectively conform closely to (i.e., remain in contact with) the substantial majorities or entireties of these surfaces through ordinary body positions and movements. Increasing the amount of elastic stretch capability imparted to gusset increases the variety of body shapes and movements to which the gusset can conform in use. In connection therewith, reducing the longitudinal tensile modulus (longitudinal tensile force in the material resulting from longitudinal stretching) tends also to increase body conformity and enhance comfort.

For underwear, many women prefer brief pants rather than legged pants such as “boy shorts” styles for ordinary daily wear. This is due to issues of comfort; unlike a legged, shorts-type pant, a brief pant ordinarily will not ride up and bunch about the legs from changes of body position, and thereby be a source of unwanted concentration of material bulk, tightness about the legs or other discomfort under outer clothing. Further, due to the manner in which their leg edges tend to cause the pant to fit through the crotch region of the body, brief pants having suitable elastic stretch characteristics may be preferred for maintaining a close fit about the female genital/urethra area, for purposes of protecting against leakage of menstrual fluid, vaginal fluid, or unintended discharges of urine.

It has been learned that a durable absorbent pant may be designed that more reliably and comfortably holds an absorbent structure against/in contact with a wearer's body through the crotch region, in a position to better intercept adhering flow, capture the discharged liquid in the absorbent material before it can escape, and permit expansion of the absorbent components, initially and over a reasonable duration of wear/use of the pant. In order for a fabric or web of material to conform to a non-ruled surface, it must be capable of stretching along at least one direction. It has been determined that the direction of stretch that most effectively enables a fabric or web of a pant garment to effectively conform to the contours of the female lower torso and crotch region is, substantially, the longitudinal direction.

Referring to FIGS. 1 and 2 as an illustrative but non-limiting example, a pant may include a front waist portion 100, a rear waist portion 120 and a crotch portion 130 bridging the front and rear waist portions. Front waist portion 100 has a front waist edge 102, and left and right front leg opening edges 104f. Rear waist portion 120 has a rear waist edge 122, and left and right rear leg opening edges 104r. Crotch portion 130 has left and right crotch leg opening edges 104c. Crotch portion 130 may include the entirety or a portion of a gusset 230 that may include several layer components that will be described further below. (For purposes herein, the “crotch portion 130” is a portion of a pant identified as described herein, and its forward and rearward extents are independent of specific components or structures.) Herein, a “gusset” 230 is a structural component that includes at least two distinct layers including an absorbent layer and a liquid impermeable barrier layer, and is at least partially disposed in the crotch portion and may bridge the front waist portion 100 and the rear waist portion 120. A gusset has a “liquid impermeable barrier layer” if it exhibits z-Direction Leakage no greater than 0.1 ml of test fluid into a section of filter paper, in the Liquid Impermeability test described below. Referring to FIG. 2 by way of example, for purposes herein, crotch portion 130 is the portion of the pant, at a minimum, lying between crotch portion minimum front extent 401 and crotch portion minimum rear extent 402. Crotch portion 130 is longitudinally centered about crotch portion lateral axis 301 (which is drawn along the smallest width dimension measured between the crotch leg opening edges 104c), and includes 10 percent of the overall length L of the pant to the front and the rear of the crotch portion lateral axis 301. As suggested by FIG. 2, it may be desired that the crotch portion lateral axis 301 be disposed forward of the lateral axis 300 (which equally divides overall length L), rather than be co-located with lateral axis 300, for purposes of better fit about an adult female wearer's legs and lower torso. Thus, the respective boundaries 401, 402 between crotch portion 130 and front and rear waist portions 100, 120 for purposes herein, are independent of the location(s) of any seams such as seams 134, 138 that may be present to join material(s) included in the gusset 230 and material(s) included in the front and rear waist portions 100, 120. Material forming the front and rear waist portions 100, 120 may be integral and continuous across, or may be joined at seams, at or proximate hip portions 160, to complete a pant structure having a waist opening and a pair of leg openings as illustrated in FIG. 1.

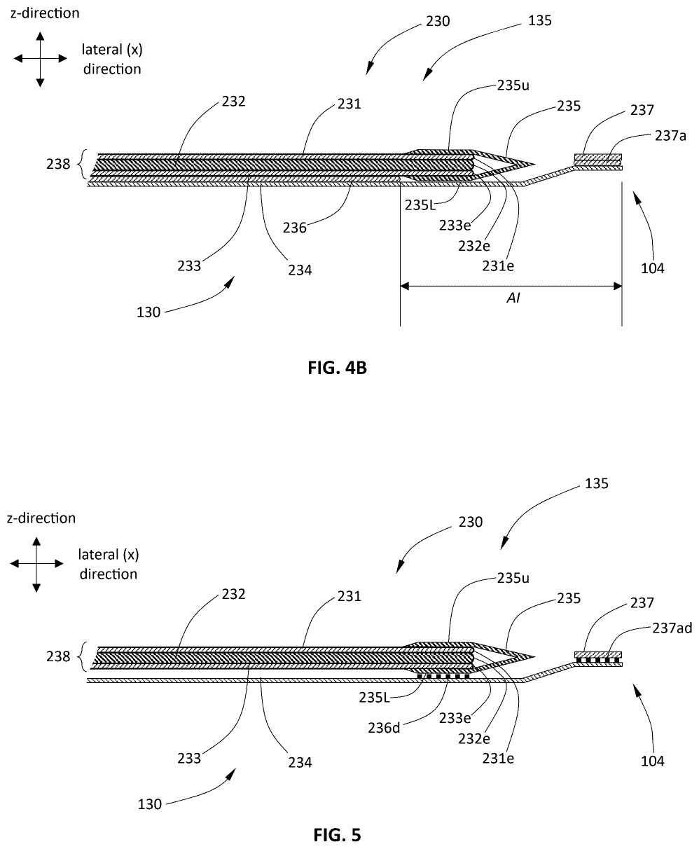

Material(s) forming one or both of forward and rearward portions 132, 136 of crotch portion 130 may be integral and continuous with material(s) forming front and rear waist portions 100, 120, or alternatively, one or both of forward and rearward portions 132, 136 of crotch portion 130 and/or gusset 230 may be substantially formed of one or more sections or layers of material that are distinct from material(s) substantially forming one or both of front and rear waist portions 100, 120, and gusset 230 may be joined to front and rear waist portions 100, 120 at one or both of forward seam 134 and rearward seam 138. In illustrative but non-limiting examples suggested in FIGS. 1, 2 and 10A-10C, front waist portion 100, an outward-facing layer 234 of crotch portion 130, and rear waist portion 120, may all be formed partially or entirely of a first single, continuous section of material, e.g., the fabric from which the main body of the pant is formed. As reflected in the examples shown in FIGS. 4A, 4B, 5, 6A-6C, 7A-7C, 8, 9 and 10A-10C, one or more additional layers of material 231, 232, 233 may be disposed over the wearer-facing side of outward-facing layer 234 in crotch portion 130 as shown, and be connected or affixed directly or indirectly to layer 234 via, e.g., stitching/sewing, adhesive bonding, thermal bonding (fusing or welding) or other suitable attachment/joining mechanism (hereinafter, attachment mechanism) at, e.g., forward and rearward gusset seams 134, 138 and/or along the crotch leg opening edges 104c.

In other non-limiting examples (not specifically shown), the sections of materials respectively forming front waist portion 100, rear waist portion 120 and crotch portion 130 may be entirely separate and distinct, and joined via any suitable attachment mechanism at, e.g., forward and rearward seams 134, 138. This configuration may be preferred in some circumstances because it may provide the designer with greater flexibility in selection of the respective materials for the waist and crotch portions with respect to appearance, feel, weight, breathability, elongation, stretch characteristics and cost. The elongation and stretch characteristics described below would be applicable to the one, or more layers of material in combination, present in crotch portion 130 and/or gusset 230.

### Crotch Portion/Gusset

### Gusset Components

Referring to FIGS. 3A, 3B and 3C, a gusset 230 includes components of an absorbent assembly 238 which include, at a minimum, an absorbent layer 232 and a barrier layer 233. The absorbent layer 232 may be one or more layers of suitable fabric, selected for desired absorbency and stretch characteristics. Absorbent layer 232 will be underlaid by a barrier layer 233, which may be a suitable polymeric film, or alternatively, a fabric that is effectively resistant to passage of liquid therethrough under ordinary conditions of wear of garments of the type described herein. Barrier layer 233 serves to prevent liquids absorbed by the absorbent layer 232 from passing by wicking or capillary action out to an outward-facing layer of the pant, and from there out to outer clothing, bedclothes, etc.

In some examples (not specifically shown), absorbent layer 232 may include or be formed of two, three or more distinct sublayers of material disposed superadjacent/subjacent one another. The respective sublayers may have different sizes, dimensions and/or surface areas along the x- and y-directions, and may be formed of or include respectively differing materials. In some examples, one or more of the sublayers may be formed of or include a nonwoven web material. In such examples, for reasons explained herein, it may be preferred that such sublayers be constituted and manufactured so as to possess elastic extensibility, or alternatively, be arranged within the gusset so as to be unbonded/unattached to superadjacent or subjacent layers (e.g., elastically extensible layers), over a majority of the overlapping surface area therebetween, to avoid impeding elastic stretch of such superadjacent/subjacent layers.

Barrier layer 233 may be formed of or include a suitable polymeric film. The polymer component(s) of the film may be selected so as to impart one or both of effective liquid impermeability and elastic extensibility thereto. In some examples, the film may be adapted to be liquid impermeable under ordinary conditions of use contemplated herein, but be effectively gas and/or vapor permeable (i.e., “breathable”), so as to allow water vapor to more easily escape the garment, and thereby improve comfort and reduce chances for overhydration of the wearer's skin (which can cause or contribute to undesirable growth of microorganisms, skin irritation, rash, etc.). Without intending to be bound by theory, it is believed that such breathability may also contribute to reduction of malodor, by permitting venting of low-molecular-weight VOCs and/or by reducing water content in the gusset, thereby making absorbed matter drier and less hospitable to growth of microorganisms. In some examples, it may be preferred that barrier layer 233 be unattached to overlying absorbent layer(s) in middle/central regions of the gusset between its peripheral edge seams, to reduce binding, stiffening and elasticity-reducing effects of bonding or affixing superadjacent/subjacent layers.

In some examples gusset 230 may include an additional, overlying wearer-facing layer 231. Wearer-facing layer 231 may be formed of a suitable fabric that has a comfortable feel against the skin while being configured to function to conduct liquids down to the absorbent layer(s) 232 following a discharge, and subsequently, to isolate the absorbed fluids in the absorbent layer from the wearer's skin, to help impart a relatively dry feel to the pant for the wearer, even after a discharge.

In most designs it may be preferred to provide edge sealing of the absorbent layer 232 and wearer-facing layer 231 (if included), about the perimeters of these layers, e.g., about a portion or the entirety of the perimeter of the gusset 230, or at least at the perimeter edges of the gusset proximate the leg opening edges 104c in the crotch portion 130 of the pant. Since these layers may be formed of fabrics configured to accept and transfer liquid via wicking or capillary action, it may be desired to block liquid movement out from the perimeter edges of these layers, which can result in leakage and soiling of outward-facing layer 234 of the crotch portion, outer clothing, bedclothes, etc.

Referring to FIGS. 3A and 3B, in some examples, edge sealing may be provided by including an edge sealing strip 235. Edge sealing strip may be formed of a film or fabric that is suitably waterproof or effectively resistant to passage of liquid therethrough, under conditions of use contemplated herein. Edge sealing strip 235 is preferably sealingly adhered to barrier layer 233 so as to be effectively unitary therewith, and wraps up and around the perimeter edges of the absorbent layer(s) 232 and wearer-facing layer 231 (if included), and is affixed to the wearer-facing surface of the uppermost layer proximate the perimeter thereof. So configured, edge sealing strip can be effective to block passage of liquid that may wick to the perimeter edges of these layers, out therefrom. Edge sealing strip 235 may be formed of an inherently tacky material having sufficient adhesive properties to enable it to adhere to barrier layer 233 and wearer-facing layer 231 with or without application of heat and pressure, or alternatively, edge sealing strip 235 may be adhered to these component via a suitable adhesive. For purposes of retaining any inherent elastic stretchability of individual layers 231, 232, 233 in the assembly, it may be desired that edge sealing strip 235 be formed of an elastomeric material. In some examples, edge sealing strip 235 may be formed of a substantially liquid impermeable polymeric film having partially or predominantly a silicone (polysiloxane) component, which may be preferred for its relative softness, pliability, elasticity/elastic extensibility, durability and resilience.

In other examples such as suggested in FIG. 3C, edge sealing may be provided by an integral, continuous extension of barrier layer 233, providing a perimeter margin portion thereof of a size sufficient to wrap up and around the perimeter edges of the absorbent layer(s) 232 and wearer-facing layer 231 (if included), and be affixed via adhesive or other suitable mechanism to the wearer-facing surface of the uppermost layer proximate the perimeter thereof. So configured, such wrapping margin portion(s) of barrier layer 233 can be effective to block passage of liquid that may wick to the perimeter edges of the absorbent layer(s) 232 and wearer-facing layer 231 (if included), out therefrom.

### Caliper

For purposes of minimized bulk, it may be desired that the combination of layered materials present in the crotch portion 130 and gusset 230 be selected and configured so has to have a central z-direction Caliper not exceeding about 6 mm, more preferably not exceeding about 5.5 mm, and even more preferably not exceeding about 5 mm. (Herein, the “central z-direction Caliper” or “central Caliper” is measured at a location in the crotch portion 130 corresponding with the intersection 250 of the longitudinal 200 and lateral 300 axes of the pant (shown in FIG. 2), when opened and laid out flat on a horizontal planar surface.) Based on disclosure herein and teachings available in the art concerning textiles, persons of ordinary skill in the art will be equipped to select a combination of suitable materials to create a structure in the crotch portion 130 having such Caliper, along with other features and attributes described herein.

### Absorption Capacity

In order for a durable absorbent pant to provide suitable absorbency and protection against leakage of menstrual fluid or unintended discharges of urine over a reasonable duration of wear, for a woman experiencing menstruation or light to moderate incontinence, while balancing concerns for limiting caliper/bulkiness of the crotch portion while limiting the required relative planar size/surface area of an absorbent portion, it may be desired that a combination of material forming the structure within the crotch portion 130 of the pant have an Area Absorption Capacity from 0.1 ml/cm² to 0.4 ml/cm², and a Volume Absorption Capacity from 0.4 ml/cm³ to 1.0 ml/cm³, measured according to the Absorption Capacity Measurement method set forth below. Based on disclosure herein and teachings available in the art concerning textiles, persons of ordinary skill in the art will be equipped to select a combination of suitable materials to create a structure in the crotch portion 130 having such absorption capacity.

### Elastic Stretch Attributes

Through experimentation it has been learned that imparting a pant with appropriate directional elastic stretch characteristics within the crotch portion 130 are important to provide a pant with a crotch portion that is both comfortably and securely held against the wearer's body surfaces, in position to intercept and absorb an adhering flow of menstrual fluid or urine, and thereby prevent leakage from the pant, through normal ranges of body positions and movements. It has been learned that the combination of materials present in the crotch portion 130 preferably should exhibit a maximum Longitudinal Elongation of 25 percent to 100 percent, measured according to the Maximum Elongation Measurement Method set forth below. Alternatively, or additionally, it may be desired that the combination of materials present in the crotch portion 130 exhibit a Longitudinal Tensile Modulus, measured according to the Longitudinal Tensile Modulus Method set forth below (reflecting the presence of one or more materials that impart elasticity) of 10 gf/mm to 100 gf/mm. Based on disclosure herein and teachings available in the art concerning textiles, persons of ordinary skill in the art will be equipped to select a combination of suitable materials to create a structure in the crotch portion 130 having such longitudinal elongation and tensile modulus properties.

### Materials Selection

Generally, the front and rear waist portions 100, 120 of suitable examples of a pant may be formed of any fabric material or combination of fabric and other materials known and used as components of underwear, swimwear or athletic/active wear, exhibiting suitable attributes that may include, depending upon the location of the fabric within the structure, pleasing feel against the skin (softness and/or low-friction/smooth/silky feel), low caliper/bulk, elongation capability, elastic extensibility, absorbency, wicking ability, breathability, etc. In one example, the front and rear waist portions 100, 120, and the outward-facing layer 234 of the gusset 230, may include or be formed of a knitted stretch fabric, in some examples, a combination of one or more of nylon, polyester, cotton, rayon/viscose and elastomeric fibers such as elastane fibers (e.g., LYCRA SPANDEX (a product of The Lycra Company, Wilmington, DE)).

Additional layer components of crotch portion 130 and/or gusset 230, as herein described requiring absorbency and longitudinal elastic stretch attributes, may include a combination of several materials selected to impart the structure with the desired attributes.

### Fabric Structure

### Most Durable Fabrics Exhibit Anisotropic Elongation Capabilities.

Woven fabrics, formed by weaving, are formed of two groups (warp group and weft group) of interlaced constituent yarns or threads, the yarns or threads within each group being substantially parallel to each other, and substantially perpendicular to the yarns or threads in the other group, along the plane of the fabric. Unless the constituent yarns or threads are themselves formed of extensible material, woven materials have relatively low elongation capabilities along the warp and weft directions, and have relatively higher elongation capabilities along the two bias directions approximately 45 degrees from the warp and weft directions. Consequently, where it is desired for particular reasons that a layer component of the crotch portion 130 and/or gusset 230 be a woven material, it may be desired that the material be oriented within the crotch portion such that one of the warp and weft directions is oriented from approximately 30 degrees to approximately 60 degrees, preferably from approximately 38 degrees to approximately 52 degrees, and more preferably approximately 45 degrees from the longitudinal direction of the pant, so as to provide maximum available longitudinal elongation capability for the woven layer.

However, when stretched along the bias direction, woven fabrics typically exhibit a substantial Poisson effect contraction (sometimes called “necking”) along the trans-stretch direction (90 degrees from the stretch direction). When such a fabric is included in the crotch portion 130 with its bias oriented approximately along the longitudinal direction of the pant, the Poisson contraction effect may cause the crotch portion to laterally narrow, which may be deemed undesirable when effective coverage of the wearer's body through the crotch region is desired.

The constituent yarns or threads of knitted fabrics, by contrast, do not follow straight paths along the plane of the fabric, and are neither parallel nor perpendicular to each other. Rather, each constituent yarn or thread of a knitted fabric follows a looping path along successive rows, interlooping with one or more constituent yarns or threads in adjacent rows. As a consequence, knitted fabrics exhibit relatively greater elongation capability along all directions as compared with woven fabrics, even where the constituent yarns or threads themselves are not extensible. For this reason, unless a woven fabric is desired for a particular reason, it may be preferred that a knitted fabric be used to form any one or more, or all, of the fabric layers present in the crotch portion 130 and gusset 230 of the pant.

Even so, most types of knitted fabrics have elongation capabilities that are anisotropic along the plane of the fabric, having a first direction of greatest elongation capability and a second direction, perpendicular to the first direction, of least elongation capability. Accordingly, when knitted fabric is selected and used to form one or more layers present in the crotch portion 130 and gusset 230 of the pant, it may be desired that the fabric(s) forming any, some or all of the layers be oriented such that their directions of greatest elongation capability are at least approximately parallel with the longitudinal direction of the pant.

In some circumstances it may be desired that a knitted fabric selected to form a layer be a rib knit type. Rib knitted fabrics exhibit relatively high elongation capability along a direction parallel to the knit rows (perpendicular to the “ribs”), with relatively low Poisson contraction effect along the trans-stretch direction. Thus, in some circumstances, it may be desired that one or more layers present in the crotch portion 130 and gusset 230 of the pant be formed of a rib knit fabric, with the “ribs” oriented substantially along the lateral direction of the pant.

In some circumstances it may be desired that the absorbent layer 232 be formed of either woven or knitted terrycloth, for purposes of increasing aggregate fiber surface area and capillarity per unit fabric surface area, and thereby, providing increased absorbency to the absorbent layer 232, while still providing a durable fabric, in contrast to a nonwoven batt or matt of fibers. In conjunction therewith or as an alternative, and for purposes of enhancing absorbency, it may be desired that constituent fibers of the yarn(s) or thread(s) from which the absorbent layer fabric material is knitted or woven be in the form of microfibers (i.e., fibers having an average denier of one (1) or less). It may be further desired that the constituent fibers be split microfibers. Yarns or threads formed of microfibers, particularly split microfibers, provide relatively greater fiber surface area per unit yarn/thread denier. When the fiber surfaces are hydrophilic, this imparts relatively greater absorbency to the fabric.

### Fabrics Constituent Yarn/Thread Compositions

As contemplated herein, wearer-facing 231 and absorbent 232 layers present in the crotch portion and gusset of the pant are expected to be exposed to discharges of urine, vaginal fluid, and/or menstrual fluid, and are expected to receive, absorb and retain these fluids for a reasonable duration of wear time, preferably while leaving the wearer-facing surfaces as dry-feeling as possible. It may be desired that a wearer-facing layer 231 have a soft feel against the skin.

Accordingly, it may be desired that the wearer-facing layer be formed of a material that has a soft feel and has suitable wicking attributes so as to efficiently conduct discharged liquid to an absorbent layer beneath, while having minimized tendency to retain liquid and thereby have a wet feel for the wearer. Thus, it may be desired that a wearer-facing layer 231 not include a predominant proportion of fibers typically used as absorbers, e.g., cotton or other plant fibers, or rayon fibers. (For purposes herein, the term “rayon” includes rayon, viscose, lyocell, and any other fibers spun from reconstituted/regenerated cellulose.) Preferably, the wearer-facing layer will not include any proportion of such fibers greater than 10 percent by weight of the fabric of the layer 234, and preferably, will include no substantial proportion of such fibers. Suitable materials may include polypropylene, polyesters and polyamides (e.g., nylon). Examples of these materials, when used to spin fiber components and/or when having received suitable hydrophilizing treatment, impart the spun fibers with suitable hydrophilic surface attributes (enhancing wicking), with relatively low individual fiber texture (reducing porosity and capillarity, and therefore, absorbency of the fabric). Additional materials may be incorporated in yarn or thread components for purposes of enhancing skin feel (e.g., enhancing a slick or silky feel against the skin) and/or further affecting hydrophilicity and/or reducing absorption tendencies. In some examples, polypropylene and/or polyethylene fiber components may be included for these purposes. In some examples, resins from which constituent fibers are spun may include additives to the primary polymer components, incorporated for enhancing skin feel, adjusting hydrophilicity, reducing absorbency, etc. In some examples, polyester or nylon component resins may include an additive comprising linolenic acid, to the extent of and as described in US 2017/0369698, for purposes of enhancing elongation and skin feel attributes, while reducing material usage and cost.

Although commercially processed cotton or other commercially processed plant fiber, or semi-synthetic, cellulose-derived materials such as rayon may be considered for inclusion as component material for the absorbent layer for their inherent absorbency attributes, some of these materials may have a tendency, undesirably, to retain constituents of menstrual fluid or urine following laundering. Accordingly, one or more of polyester, polyamide and/or combinations thereof may be preferred as component resins or even the main/predominant component resins from which fiber components of yarn or thread components of fabrics for the absorbent layer 232 are formed. The constituent threads or yarns of the absorbent layer may be knitted in a manner that imparts a relatively lofty and/or densely fibrous network having a degree of void volume and capillarity making it suitable for absorbing fluid. In some examples, the knit may be a knitted terrycloth, or even a knitted/sheared terrycloth. The constituent threads or yarns may be or may include microfibers, for increased fiber surface area per unit x-y surface area or volume of the absorbent assembly.

For purposes of imparting elastic extensibility to layers present in the crotch region (particularly longitudinal stretch), it may be desired that yarn or thread components of one or more of the fabrics present include elastically extensible fibers, yarns or threads. In some examples, elastically extensible fibers, yarns or threads may be formed of or include elastane or spandex (such as LYCRA, currently available from The Lycra Company, Wilmington, Delaware), which are particularly elastically extensible and durable through a plurality of launderings, as compared to other elastic materials used to elasticize fabrics.

In other examples, one or more elastically extensible polymer film layers distinct from the fabric layer(s) in the crotch portion 130 may be included to impart elastic extensibility to the structure in the crotch portion as a whole.

An elastic polymer film layer may be formed of any suitable elastic polymer material. In some examples, an elastically extensible film layer may be formed by extrusion or other application of film resin in molten or semi-molten form onto a layer component fabric, whereby the molten resin partially penetrates the fabric and upon cooling forms a film that is partially mechanically enmeshed in and/or made integral with the fabric.

### Antimicrobial Agents

For purposes of hindering growth of microorganisms supported by components of absorbed urine, vaginal discharge, or menstrual fluid, which may cause odor, it may be desired to include one or more antimicrobial agents in or among the materials present in the crotch portion 130. Any such antimicrobial agents are preferably included in a form adapted to remain in place and continue to be effective following a plurality of launderings of the pant. In some examples, an antimicrobial agent may include particles or fibers including a metal, metal alloy or metallic compound that includes one or more of copper, silver, zinc, aluminum or combinations thereof. In other examples an antimicrobial agent may include particles or fibers including carbon or a composition or compound including carbon. One or more of these materials may be included as additives to resins from which constituent fibers are spun, or may be included in compositions that are topically applied to constituent yarns, threads or fabrics following manufacture thereof. Such antimicrobial agents are preferably included in material(s) forming the absorbent layer 232 and/or the wearer-facing layer 231.

### Odor Adsorbent Structure

Odor-causing VOCs can be generated by certain microorganisms, including certain species of bacteria, whose growth and reproduction can be supported by components of menstrual fluid, vaginal discharge and/or urine, and ambient conditions including temperatures, that are proximate body surfaces covered by garments.

As noted in the Background, antimicrobial agents may be deemed undesirable in some circumstances, or otherwise may be deemed insufficient, and it may be desired to provide an alternative or supplementary mechanism to manage odor that may develop about discharged menstrual fluid that is retained by absorbent components of a pant.

As a supplement or alternative to inclusion of antimicrobial agents, it may be desirable to include an odor adsorbent material into the pant, preferably proximate the gusset. Suitable odor adsorbent materials function by binding airborne VOCs to surfaces of and/or within pores or interstices within the adsorbent material. Potential adsorbent materials may include zeolites, functionalized silicas, metal-organic frameworks, and activated carbon, and combinations thereof. In some examples, activated carbon may be preferred, for the reason that it can be readily obtained in various suitable forms, including fibers.

Activated carbon is commercially available in forms including fiber, particulates and/or granules, and can be used in dry form as an adsorbent of some types of airborne molecules, including certain airborne molecules of volatile organic compositions (VOCs).

Examples of fabric materials or laminates thereof, having activated carbon incorporated therein, or forming a component thereof, are currently available from Odegon Technologies Limited (Telford, UK). In one form, fibers of activated carbon are included in one or more types of filaments or yarns forming component(s) of a knitted or woven fabric or nonwoven web material. In another form, powdered activated carbon, together with a suitable binder, is/are applied to surfaces of a fabric or its constituent fibers or yarn(s), and held in place on the fibers or yarn surfaces via the binder. It has been learned that these materials may be incorporated into an adsorbent structure, that when suitably configured and suitably located within an absorbent pant, serve to adsorb airborne VOC molecules proximate discharged/retained menstrual fluid, and thereby manage odor. It has been learned, further, that such structure, suitably configured, can allow for a plurality of launderings or washings of the garment, with retention of the activated carbon material, and renewal and/or retention of its odor-adsorbing efficacy, following such laundering or washing. It is believed that including activated carbon fiber as a constituent of thread or yarn components of a knitted or woven fabric may be an advantageous way of ensuring that the activated carbon remains in the fabric and available to serve as an adsorbent, following a plurality of uses and launderings, as compared with other compositions/configurations (including other incorporated antimicrobial materials and other mechanisms for affixing activated carbon material onto or within fabrics), although product development continues in the field.

It has been learned, further, that providing a layer of protective fabric or film, to overlie a fabric layer that includes adsorbent material, serves to add mechanical strength and durability to the combination, and reduce mechanical/friction wear and loss of the adsorbent material in the adsorbent layer, and thereby help preserve its efficacy as an odor management mechanism through repeated wear and laundering.

An odor adsorbing structure may also include a substrate layer, wherein an adsorbent layer including adsorbent material, is affixed to a substrate. The substrate may be another fabric layer, a film layer, a layer of adhesive material, or an adhesive material in the form of a film. Adhesive material may be of a hot-melt type adhesive, whereby the odor adsorbent structure may be applied and affixed to fabric of a garment, via hot iron or hot press. In some examples, the adhesive or film adhesive may be a polyamide-based material.

A protective fabric or film layer (if included) may be affixed to an odor adsorbing layer, and an odor adsorbing layer may be affixed to a substrate layer (if included), by any mechanism suitable for affixing fabrics and/or films of garments together, including by way of adhesive film, hot-melt adhesive, micro-dot adhesive and/or sewing/stitching. Similarly, an odor adsorbing structure may be affixed to a fabric component of a pant by any mechanism suitable for affixing fabrics and/or films of garments together, including by way of adhesive film, hot-melt adhesive, micro-dot adhesive and/or sewing/stitching. In some examples an elastic adhesive film, elastic adhesive or micro-dot adhesive may be preferred, for purposes of efficiency of manufacture and minimizing impairment of stretchability of the portion(s) of the pant to which the odor-adsorbent structure is affixed.

Regardless of the attachment mechanism used, however, as described above, structures that include a fabric layer including adsorbent materials, and optionally one or more additional layers such as an overlying protective layer and/or an underlying substrate layer, may have restricted or limited elastic stretch capabilities. Thus, affixing them to a garment that is formed of elastically stretchable material(s), where such elastic stretch is desired for purposes of, e.g., comfort, snug fit and effective fluid capture/containment, may have the effect of compromising desired stretch characteristics of the garment.

Additionally, without intending to be bound by theory, it is believed that exposing an adsorbent material such as activated carbon to wetting by menstrual fluid may reduce efficacy of the material at adsorbing airborne VOCs following repeated uses and launderings. It is believed that this reduction in efficacy may result from retention of menstrual fluid constituents within pores or interstices of the adsorbent material following laundering or washing—that complete/thorough removal of such constituents via ordinary laundering or washing may be difficult. As pores or interstices within the adsorbent material become occluded or occupied by remnants of discharged fluid constituents, surface area available to adsorb airborne VOC molecules is reduced.

Further, the inclusion of an adsorbent structure (whether it includes fabric composed in part of activated carbon fibers, activated carbon material, or other adsorbent material captured and retained by another mechanism or structure) onto or into an absorbent gusset structure may interrupt and/or impair the wicking and absorbency performance of the absorbent gusset structure, making it less effective for its primary function. It will often be the intent, reflected in the selection and configuration of materials of the gusset, that the top/wearer-facing layer 231 be effective at wicking fluid so as to receive and rapidly conduct it downwardly to an underlying absorbent layer 232. It will be the intent that the underlying absorbent layer 232 be effective at drawing fluid from the top layer, and imbibing, absorbing and retaining the fluid within its structure. In order for the wearer-facing layer 231 to be situated to transfer and yield discharged fluid down to the absorbent layer, the two layers 231, 232 should be in direct face-to-face contact. Thus, if a structure is interposed between these two layers, this contact is lost and the system may have compromised effectiveness at intercepting, receiving, distributing, absorbing and containing menstrual fluid. Alternatively, adsorbent materials such as activated carbon may be hydrophilic, and thereby be absorbent themselves. If such materials are interposed between the wearer-facing layer and the absorbent layer, they may intercept fluid and retain it, while being susceptible to having it expressed therefrom under pressure imposed by wearer movements, sitting, etc.—creating a risk of undesirable rewetting of the wearer-facing layer with previously discharged fluid.

The concerns reflected above have caused attention to be given to the configuration, shape, size and placement of odor adsorbent structures within durable absorbent underwear designed for management of menstrual discharge.

Accordingly, referring to FIGS. 14B-14F, an odor adsorbent structure 55 is preferably located to the wearer-facing side of the outer layer(s) of the absorbent pant, such that it faces the wearer's skin surfaces during wear. Further, it may be preferred that the basis weight of adsorbent material present be greater in the peripheral region 40 than in the central region 41. It may be preferred that a minority fraction of the surface area of the adsorbent-containing material, or even substantially none, of such surface area, be disposed in the central region 41. This is to reduce the likelihood or extent to which a majority of included adsorbent material may be subjected to repeated wetting by menstrual fluid, upon discharge or upon longitudinally- or laterally-outward migration of fluid through the absorbent layer(s) of the gusset and away from central region 41. Even more preferably, the majority of the weight of the adsorbent material is located in the peripheral region 40 but longitudinally outward of the forwardmost and rearwardmost edges 15, 16 of the absorbent layer(s).

Thus, where the adsorbent material is present in one or more discrete odor adsorbent structures 55f, 55r, it may be preferable to locate one or more such adsorbent structures in the peripheral region 40 (e.g., as shown in FIGS. 14B-14F), and more preferable to locate one or more such adsorbent structures in the peripheral region 40, but longitudinally outward of forwardmost and rearwardmost edges 15, 16 of the absorbent layer(s) (e.g., as shown in FIG. 14C).

At the same time, however, it may be desired to locate and dispose the weight majority of the adsorbent material present as closely as is practical to the locations at which absorbed fluid is most likely to be retained, to maximize the likelihood that airborne VOC molecules that may be emitted will be proximate and likely adsorbed by the adsorbent material. For this reason, it may be desired that the weight majority of the adsorbent material be disposed in the near peripheral region 40a, proximate the end edges 15, 16 of the absorbent layer(s) and not longitudinally outward thereof. Referring to FIG. 14A, the “near peripheral region 40a” is that sub-portion of peripheral region 40 that is bounded at its longitudinally outermost extents by lateral lines 35, 36, which respectively equally divide the distance between the forwardmost and rearwardmost end edges 15, 16 of the absorbent layer(s) of the gusset, and the front and rear waist edges 102, 122.

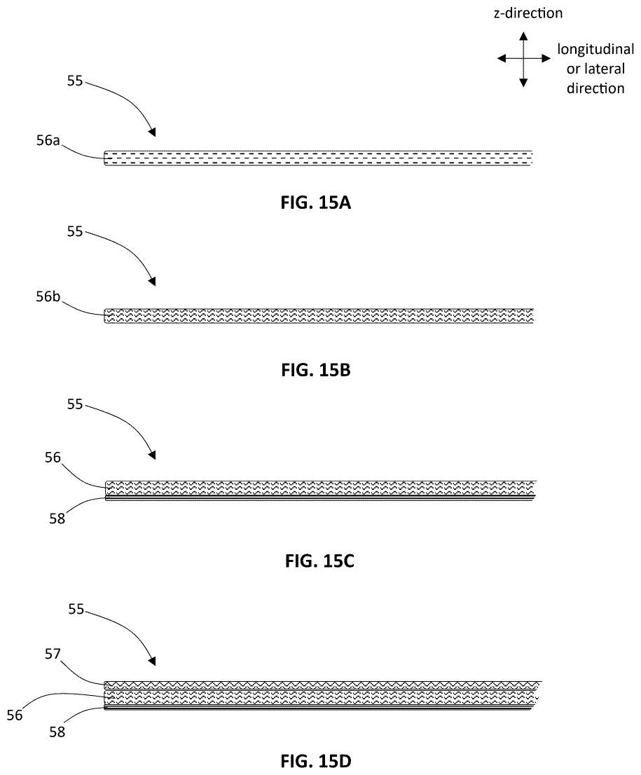

Referring to FIG. 15A, an odor adsorbent structure 55 may consist of a single odor adsorbent layer 56a, which may be formed of layer of fabric or other carrier web that has had its surfaces, or constituent fibers or yarns, treated with an adsorbent material such as activated carbon in particulate or granular form, together with a suitable binder to bind the particles or granules to the fabric or fiber surfaces. Preferably, the binder is formulated to be gas and vapor permeable so as to allow airborne VOC molecules to freely reach the pores and/or interstices of the adsorbent particles or granules. Referring to FIG. 15B, in another alternative, odor adsorbent structure 55 may consist of a single layer of odor adsorbent fabric 56b that has activated carbon fibers forming, or incorporated into, its constituent threads or yarns.

Referring to FIG. 15C, an odor adsorbent layer 56 (such as a layer 56a or 56b as described above) including activated carbon and/or other adsorbent material may be affixed to a substrate 58. Substrate 58 may be another layer of fabric included to add mechanical strength, or may be a film or layer of adhesive, adhesive in film form, or adhesive in micro-dot form as described herein. The adhesive may be a hot-melt adhesive. Such hot-melt adhesive may be a polyamide-based adhesive. Where a hot-melt adhesive is provided, it may be used to adhere odor adsorbent structure 55 to an underlying layer or fabric of the absorbent pant, via hot pressing or hot ironing to activate the adhesive.

Referring to FIG. 15D, a protective layer 57 may be included with the odor adsorbent structure 55, overlying the adsorbent layer 56. The protective layer 57 may be a layer of fabric or other gas- and vapor-permeable material (such as, for example, a vapor-permeable film, perforated or apertured film, a scrim, etc.), which will allow airborne VOC molecules to freely reach the underlying adsorbent layer 56. In some examples the protective layer 57 may be a fabric formed of knitted or woven fibers or yarns, in some examples, polyester or other polymer fibers or yarns. In some examples, the protective layer 57 may be formed of fibers or yarns that are naturally hydrophobic, or otherwise, the constituent fibers or yarns may be treated to impart hydrophobicity to their surfaces, and consequently, result in hydrophobicity of the protective layer. This may be desired to reduce likelihood that menstrual fluid, urine, or vaginal discharge will penetrate the protective layer and wet the underlying adsorbent material.

It may be desired that any uppermost wearer-facing surface of odor adsorbent structure 55 also be disposed so as to be in contact with, or directly facing, the wearer's skin. This is to maximize proximity of the adsorbent material within the structure 55 with ventilating airflow moving longitudinally outwardly from the crotch region 130 and/or gusset 230, thus maximizing likelihood that any airborne VOC molecules present in the airflow will be adsorbed by the adsorbent material.

As reflected in FIGS. 14B-14F, it may be desirable to configure odor adsorbent structures 55 such that they are elongate, with an aspect ratio of greatest dimension to least dimension of at least 2. Preferably, the greatest dimension is oriented predominantly in the lateral (x) direction. This arrangement is to provide adequate effective surface area of the odor adsorbent structure(s) 55, but minimize the restricting or limiting effect that odor adsorbent structure(s) 55 (which may themselves have limited elastic stretchability) may have on longitudinal elastic stretchability through the crotch portion 130, e.g., along the leg edges 104 (e.g., in elasticized leg bands). For the same purpose, in a further example reflected in FIG. 14E, one or more pairs of elongate odor adsorbent structures may be arranged such that the axes 55a of their longest dimensions form angles α with the proximate tangents 104t of leg edges 104 that approach 90 degrees, i.e., are from 45 degrees to 90 degrees.

If elastic leg bands or other elastic stretch features of the pant are configured to hold the pant structure close to the wearer's body and snug about the wearer's legs, this, in combination with a barrier layer of a gusset, may result in ventilation about the crotch region occurring primarily, within the crotch region, in a longitudinal direction. Accordingly, it may be desired to maximize the likelihood that ventilating airflow (which may contain airborne VOC molecules) is required to pass over or proximate the location of an odor adsorbent structure 55. Accordingly, where only a single odor adsorbent structure 55 is included either forward of the lateral axis 300 and/or rearward of the lateral axis 300, it may be desired that its lateral dimension be at least 75 percent, and more preferably at least 100 percent, of the width of crotch portion 130 at its narrowest location. Alternatively as suggested in FIGS. 14D and 14E, where two or more odor adsorbent structures 55 are arranged to laterally span the pant at a common longitudinal location, it may be desired that their combined lateral dimensions are at least 75 percent, more preferably at least 100 percent, of the width of crotch portion 130 at its narrowest location.

It is contemplated that any combination of any of the particular, single odor adsorbent structure sizes and placements described above and reflected in FIGS. 14B-14F may be configured. Thus, for example, the configuration and placement of odor adsorbent structure 55f as shown in FIG. 14B may be included singularly/alone in a pant, or in another example, combined with the configurations and placements of one or both pairs of odor adsorbent structures 55r shown in FIG. 14E.

For purposes of balancing the competing objectives, of odor management efficacy, and economy of materials usage, it may be desired that the total x-y plane surface area of adsorbent layer(s) 56 of included odor adsorbent structure(s) 55 be from 2.5 percent to 400 percent of the total x-y plane surface area of the wearer-facing surface(s) of all absorbent layer(s) 232 of the gusset (including all wearer-facing surface areas of all absorbent layers present). This proportionality may be desired because, it is believed, quantities of airborne VOC molecules that may accompany discharged, absorbed and retained menstrual fluid may be proportional to the volume of absorbed fluid present, which in turn, may be related to the size and absorbent capacity of the gusset. As an alternative measure, where the adsorbent material includes activated carbon, it may be desired that the weight of activated carbon that is included in adsorbent layer(s) 56 of included odor adsorbent structure(s) 55 be at least 0.05 mg activated carbon per square centimeter total surface area of the wearer-facing surface(s) of all absorbent layer(s) 232 included in the gusset (including all wearer-facing surface areas of all absorbent layers present).

For the purpose of not imparting to the pant undesirable bulk or structure that may cause discomfort and/or present bulges or lines visible through outer clothing, it may be desired that one or more, or all, odor adsorbent structures included in the pant have a caliper no greater than 5 mm, preferably no greater than 4 mm, and more preferably no greater than 3 mm.

In some examples, it may be desired to ensure that the odor adsorbent structure is visibly apparent to a viewer observing the inside surfaces of the pant. This, combined with a presentation of suitable information associated with the product, can help provide comfort/reassurance to the user/wearer that the pant has odor control features. In some examples, fabric(s) or other materials surrounding the odor control structure may be imparted, via dye and/or pigment, with a color that visibly contrasts with the visible color of the odor control structure. In some examples, one or more layers of the odor control structure that are visible to the user, may be imparted, via dye and/or pigment, with a color that visibly contrasts with surrounding materials of the pant.

### Barrier Layer

Where urine, vaginal, or menstrual fluid is absorbed by a fabric layer in the crotch portion 130 and gusset 230, it may be desirable to include a barrier layer, e.g., barrier layer 233 (see, e.g., FIGS. 3A-3C) to prevent the absorbed fluid from passing from the absorbent layer to an outward-facing layer 234 or even to outer clothing. In some examples, a barrier layer 233 may be formed of or include a suitable liquid impermeable polymer film. In some examples, the film composition may be selected to have elastic extensibility, providing or complementing suitable elastic stretch attributes to the combination of layers present in the crotch portion 130 and/or gusset 230. In some examples, a barrier layer 233 may be formed of or include a film formed in whole or in part of a polyurethane- or polyester-based resin. In some examples, a barrier layer 233 may be formed of or include a film that is formed by extrusion or other application of thermoplastic film resin in molten or semi-molten form directly onto the outward-facing surface of an overlying layer, such as absorbent layer 232, such that the film resin, while still molten, partially penetrates the fabric and thereby forms a liquid impermeable film that is partially mechanically enmeshed in and/or made integral with the fabric of the overlying layer. This also has the effects of consolidating these layers, which can reduce caliper in the crotch portion 130, and reducing or preventing wrinkling or bunching of the absorbent layer 232 upon elastic contraction. In a particular example, a polyurethane or polyester film may be formed by extrusion or other application of molten thermoplastic resin directly onto an outward-facing surface of a fabric that serves as or forms a component of absorbent layer 232. If the barrier layer 233 is separately formed, however, it may be desired to affixed it to the overlying layer, e.g. absorbent layer 232, via a suitable bonding mechanism, e.g., an adhesive disposed between the barrier layer and the overlying layer. A configuration that allows the barrier layer and the overlying layer to separate, so as to result in a void space between them in some circumstances, may be deemed undesirable because this may allow fluid to pool in the void space and thereby be uncontrolled and/or unmanaged by an absorbent structure, creating added risk of leakage. On the other hand, if the barrier layer 233 is not unified with an overlying fabric layer between peripheral regions of the gusset, the gusset may have enhanced pliability and stretchability since there is no binding effect resulting from integration of these layers. This latter construction may be preferred in circumstances in which, for example, management of only light menstruation or light incontinence is required, or alternatively, where the design of the garment otherwise has features that minimize the likelihood of void space between the barrier layer and the overlying absorbent layer(s).

The material selected for the barrier layer 233 may also be vapor permeable or “breathable” in that it can permit gas or water vapor to pass therethrough, while still being effectively liquid impermeable under ordinary conditions of the use contemplated herein, via a combination of having a porous structure for vapor permeability, but sufficiently small pore sizes and surfaces having low wettability (e.g. hydrophobic surfaces), for liquid impermeability. Various liquid impermeable, vapor permeable films and other materials are known and used in fields including personal hygiene and wound dressing applications. A liquid impermeable but vapor permeable barrier layer may be preferred in some circumstances for purposes of venting water vapor to improve wearer comfort and/or help avoid overhydration of the wearer's skin.

In some circumstances including those described herein (wherein, for example, the barrier layer forms a part of an edge sealing structure), it may be desired that the material(s) selected to form the barrier layer 233 be, or be processed to have, hydrophobic surfaces.

### Gusset Seaming to Outer Pant Structure

The layers forming the gusset 230, e.g., wearer-facing layer 231, absorbent layer 232 and barrier layer 233, may be joined together to form an assembly, by any suitable mechanism. In some examples, these layers may be joined about their perimeters by a suitable adhesive interposed therebetween (not specifically shown). In some examples, these layers may be joined about their perimeters by stitching. If stitching is included for this purpose, it may be desired that the stitching not penetrate barrier layer 233 or an edge sealing structure such as edge sealing strip 235—to avoid creating liquid leakage pathways.

The following objectives are contemplated herein: Particularly about and proximate the leg opening edges 104c in the crotch portion 130 of the pant, it is deemed desirable to retain, as much as possible, elasticity and extensibility of the pant structure; to minimize material stack-up that may cause discomfort or impart or exacerbate “panty lines” visible through outer clothing; to provide for suitably snug fit that holds the absorbent gusset closely against the wearer's body and avoids gapping at the leg openings with shifting body movements; to prevent wicking and leakage of liquid from side edges of the gusset; and to avoid puncturing the barrier layer and edge sealing structure of the gusset with, e.g., stitches, which can provide leakage pathways.

Referring to FIG. 4A, these objectives are served by the configuration illustrated. Gusset 230 including absorbent assembly 238 is affixed to the outward-facing layer 234 of crotch portion 130 by a strip or tape of adhesive 236, which may be of the type that may be heat-pressed between an outward-facing surface of the gusset (in the example shown, outward-facing surface of barrier layer 233) and outward-facing layer 234. The adhesive 236 is laterally inset from the leg opening edge 104 of the pant by an adhesive inset distance AI. Preferably, adhesive 236 does not overlap, underly or overly the lower portion 235L of edge sealing strip 235 (when present; note optional alternative gusset structure shown in FIG. 3C); its laterally outermost edge is disposed either at the edge of, laterally inboard from, portion 235L. This laterally inset positioning of adhesive 236 may be imparted along the entirety of the portion of gusset 230 proximate the nearest leg opening edge 104. This positioning reduces material stack-up in with the edge sealing strip 235, and also, removes the adhesive 236 from proximity with the leg opening edge 104, thus removing any stiffening effect and/or elasticity-reducing effect that adhesive 236 may have on the structure where it is applied. In some examples adhesive inset distance AI may have a minimum value of 2 mm, more preferably 4 mm, even more preferably 7 mm and still more preferably 10 mm along the entirety of the gusset edge proximate the leg opening edge 104. Where a range within tolerances supported by particular manufacturing methods is desired, the inset distance AI along each leg edge may be, e.g., from 2 mm to 20 mm, more preferably from 4 mm to 17 mm, and even more preferably from 7 mm to 14 mm. Where desired based upon material choice, a strip of fabric finishing trim 237 may be adhered to outward-facing layer 234 by trim adhesive 237a, to provide a more finished-looking edge to the pant structure at the leg opening edge 104. Referring to FIG. 4B, in another example, adhesive 236 may extend across the gusset to a greater extent, but still be inset from the leg opening edges by distance AI as shown. No stitching is required.

FIG. 5 illustrates another example configured to serve the objectives identified above. A pattern of discrete “micro-dots” of adhesive 236d may be disposed between the gusset 230 and the outward-facing layer 234 in any desired position, including between the lower portion 235L of edge sealing strip 235, and outward-facing layer 234. Adhesive in the form of a pattern of discrete micro-dots may be applied to a substrate material layer by, e.g. SEWFREE ON DEMAND equipment available from Bemis Associates Inc., Shirley, Massachusetts, USA. Other alternatives are commercially available, including patterns of adhesive micro-dots that are applied to transfer/release strips or sheets (paper or film) in a configuration enabling them to be transferred to fabric via iron or press. One such alternative is sold as PINBOND transfer adhesive, a product of New Textile Technologies GmbH, Balingen, Germany. A pattern of discrete micro-dots of adhesive may be an effective fastening mechanism for purposes herein in that fastening strength is provided through numerosity of discrete, closely-spaced fastening points at each of the micro-dots, while at the same time the micro-dots of adhesive do not have a substantial binding or stiffening effect on stretchable layer substrates to which they are adhered, because the substrates may stretch and flex between the micro-dots. The pattern also will typically have a substantially lower basis weight of adhesive, compared to a continuous strip or tape of adhesive. Thus, the adhesive micro-dots can be effective to adhere respective superadjacent/subjacent materials together, but also help substantially mitigate material stack up, and avoid binding, stiffening, and reducing elasticity of the substrates that they serve to adhere together. No stitching is required.

As a supplement to, complement to, or substitute for, adhesive micro-dots, the manufacturer may prefer to join materials where adhesive might otherwise be used, via thermal compression bonding or ultrasonic bonding, for example, in the form of a pattern of discrete bonds. Such bonds and a pattern thereof may be arranged and function in a manner similar to a pattern of adhesive micro-dots.

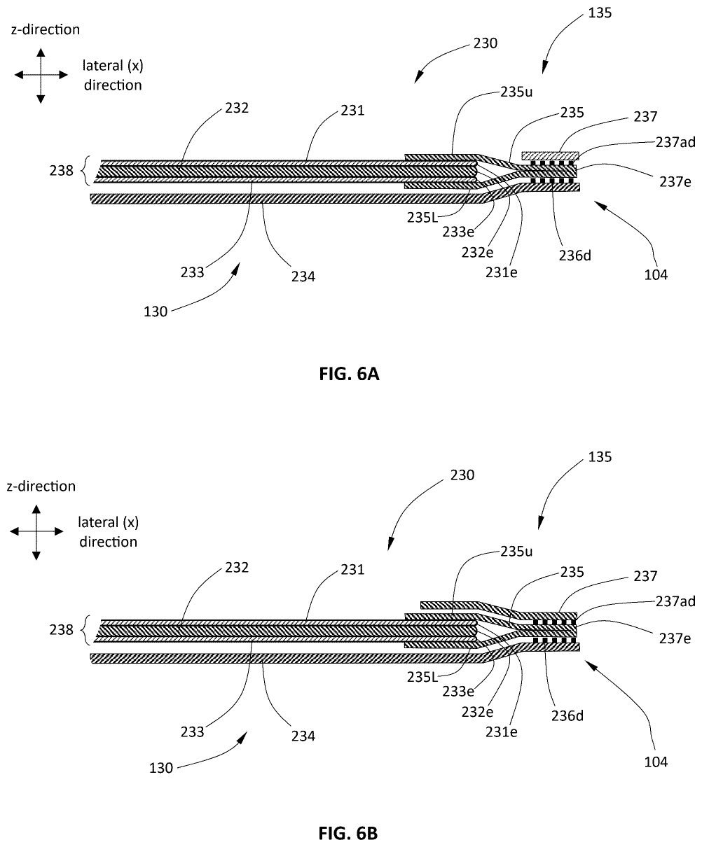

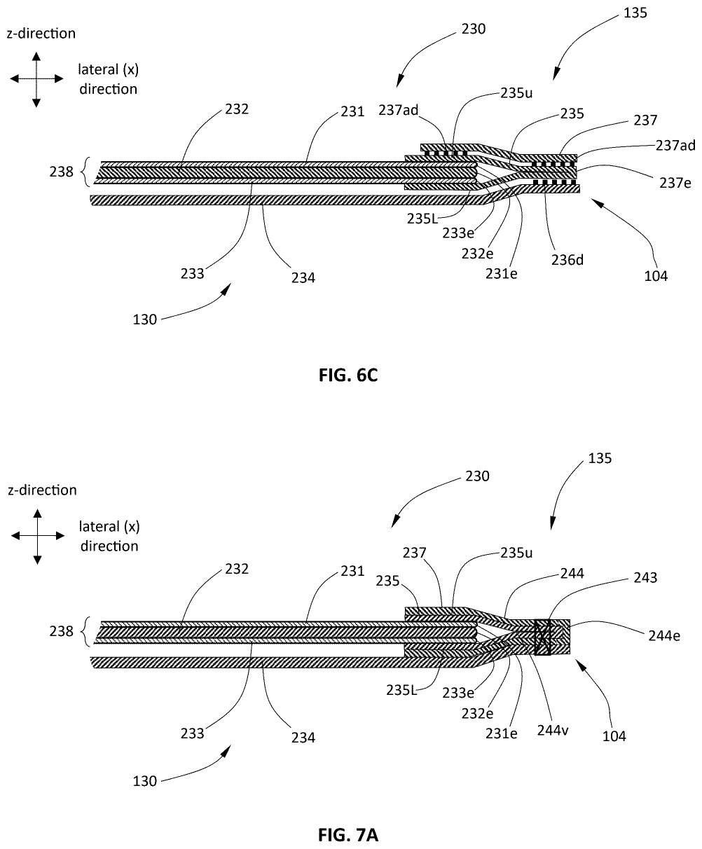

FIGS. 6A-6C illustrate additional examples configured to serve the objectives identified above. Edge sealing strip 235 is folded about itself in a “V” configuration about the peripheral edge of the absorbent assembly 238, and includes a sufficient margin of material that when pressed and adhered to itself outboard of the absorbent assembly 238 edges and proximate the fold, creates an extending portion 237e. Extending portion 237e may be attached to outward-facing layer 234 by micro-dot adhesive 236d as shown. As discussed above, the micro-dot adhesive 236d avoids material stack up, stiffening and loss of elasticity of the pant structure where it is used, in the examples shown, proximate the leg opening edges 104. These benefits may be exploited as they provide greater opportunity flexibility to include an elastically stretchable fabric trim strip 237 without substantial compromise to stretchability, elasticity and flexibility, to at least partially cover edge outer edges of sealing strip 235 and provide a finished appearance about the gusset edges. Trim strip 237 also may be adhered to the upper portion 235u of edge sealing strip by trim adhesive in micro-dot form, 237ad, as described above.

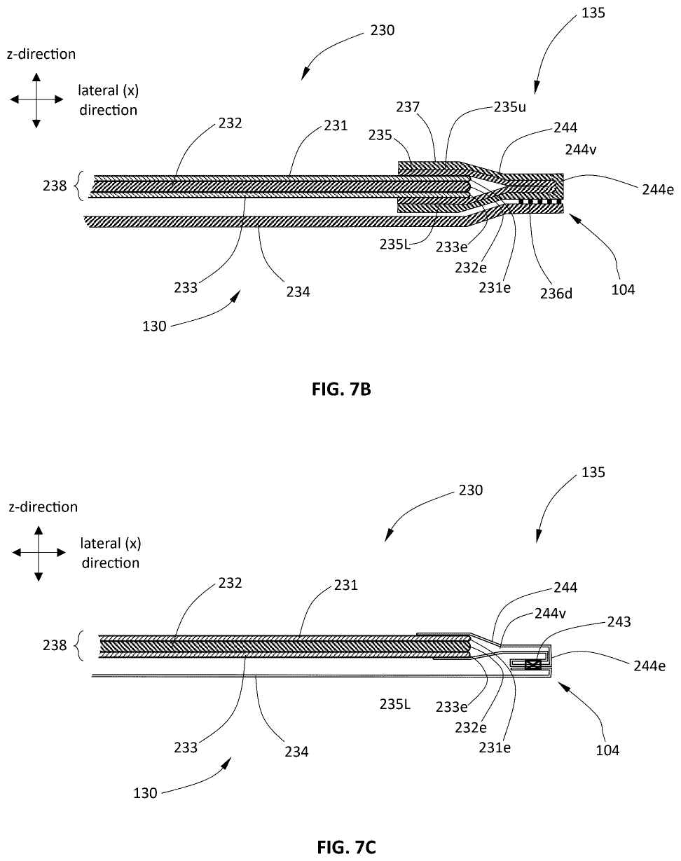

FIGS. 7A-7C illustrate additional examples configured to serve the objectives identified above. An elastic edge sealing material 235 and an extensible fabric trim material 237 may be combined as a laminate strip, which may then be folded over on itself along its length to form a combination “V”-shaped strip 244, with legs of the “V” respectively adhered to the wear-facing and outward-facing surfaces of the absorbent assembly 238. Sufficient margin may be provided outboard of the perimeter edges of assembly 238, to provide an extending flange 244e. Extending flange 244e may then be affixed to outward-facing layer 234 by any suitable mechanism, such as by stitching 243 (FIG. 7A) or micro-dot adhesive (FIG. 7B).

Where stitching is used to attach the gusset assembly 230 to the outward-facing layer 234 as suggested in FIG. 7A, the stitching is preferably of a stitch pattern that provides for extensibility along the path of the stitching. A variety of examples of potentially suitable patterns are known in the garment-manufacturing field, including, for example, zig zag stitch, lightning bolt stitch, triple straight stitch, twin needle stitch, serge stitch, blind hem stretch stitch, honeycomb stitch, feather stretch stitch, etc. It may be appreciated from FIG. 7A that, although the stitches penetrate the edge sealing material 235, the margin of extra material of the laminate 244 enables the edge sealing material 235 component thereof to be adhered to or be in sealing contact with itself inside the “V” structure, at a location 244v inboard of the stitching 243, thereby eliminating leakage pathways to the stitching 243.

In another example illustrated in FIG. 7B, the gusset 230 may be affixed to outward-facing layer 234 with micro-dot adhesive 236d, as described above.

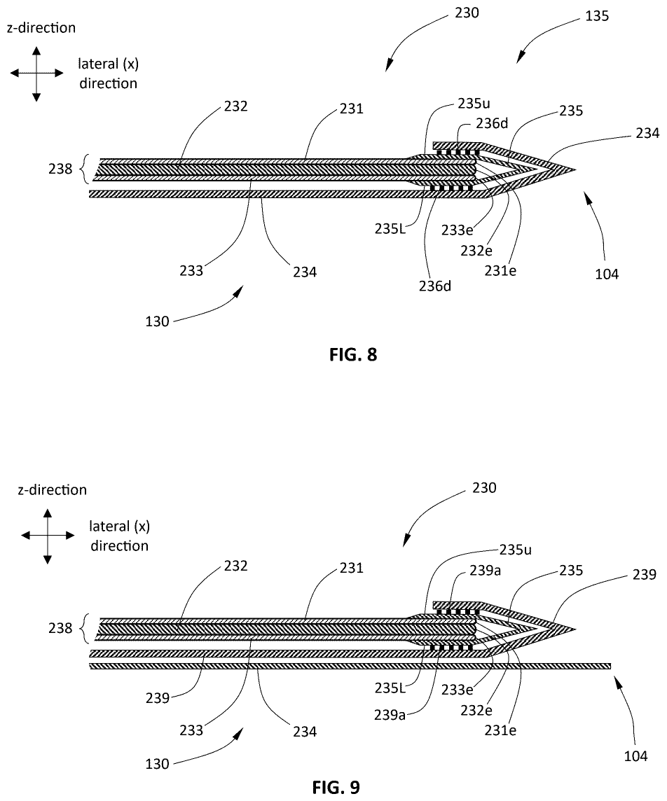

FIG. 7C, simplified for purposes of visual clarity, illustrates another possible example, in which a seam 135 formed by stitches 243 joining gusset assembly 230 and outward-facing layer 234 together are concealed. This configuration may be formed by first stitching gusset 230 through extending flange formed of laminate 244, to outward-facing layer 234 along leg edges 14, while each are in an inverted or “inside out” configuration relative each other, while leaving the forward and rearward ends of the gusset unattached to the outward-facing layer. Next, the affixed gusset and outward-facing layer may be inverted, or turned “outside out,” to result in the configuration reflected in FIG. 7C, which internalizes and conceals the stitching 243 and presents a leg edge 104 having a hemmed and finished appearance. Although this configuration may appear to have an increased stack up of materials proximate the leg edges 104, selection of relatively low caliper, lower basis weight materials for the fabric trim material layer of laminate 244 can have a mitigating effect. Further, as can be seen, the materials forming the absorbent assembly 238 of the gusset do not underly or overlie the seam 135 joining the gusset 230 to the outward-facing layer 234 proximate the leg edges 104, and so do not contribute to material stack up at the leg opening edges. (This avoidance of stack up at the leg opening edges, by absorbent assembly 238, is generally true for all examples shown and described herein—in all examples, the perimeter edges of absorbent assembly 238, i.e., perimeter edges of layers 231, 232 and 233, are disposed laterally inboard of the leg opening edges 104.) FIG. 8 reflects yet another approach. In the reflected configuration, an extra margin of outward-facing layer 234 is provided to wrap up and fold around the folded perimeter edge of edge sealing strip 235, to provide a leg opening edge 104 having a finished appearance. Deposits of micro-dot adhesive 236d may be used to adhere the gusset 230 to the outward-facing layer 234 one or more of the locations shown and/or other locations, but at least at the upper location shown, proximate the wrapped edge of outward-facing layer 234.

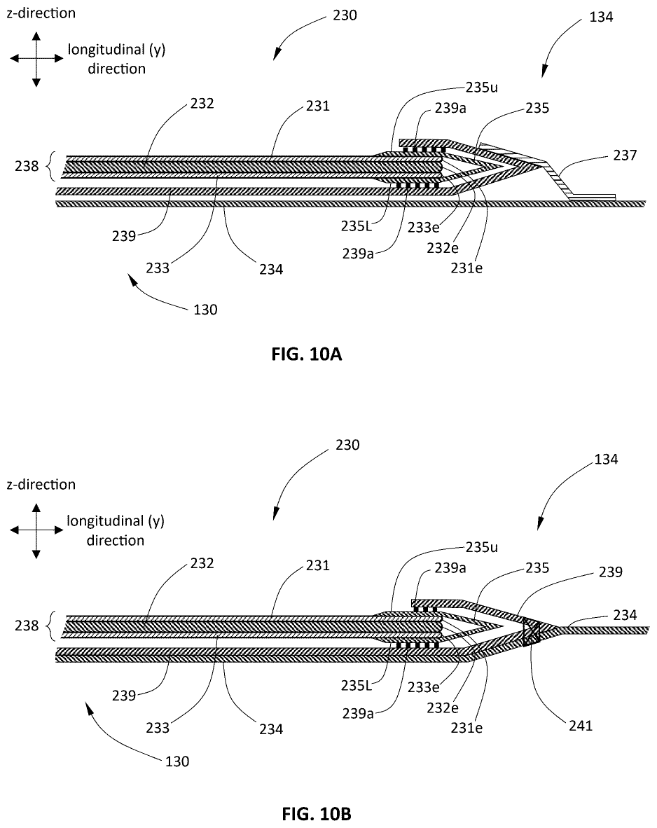

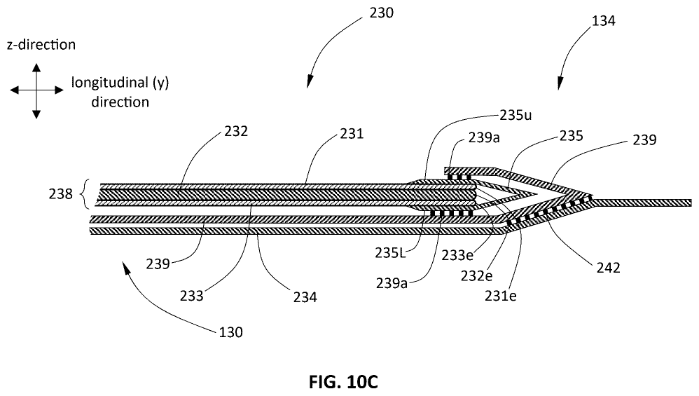

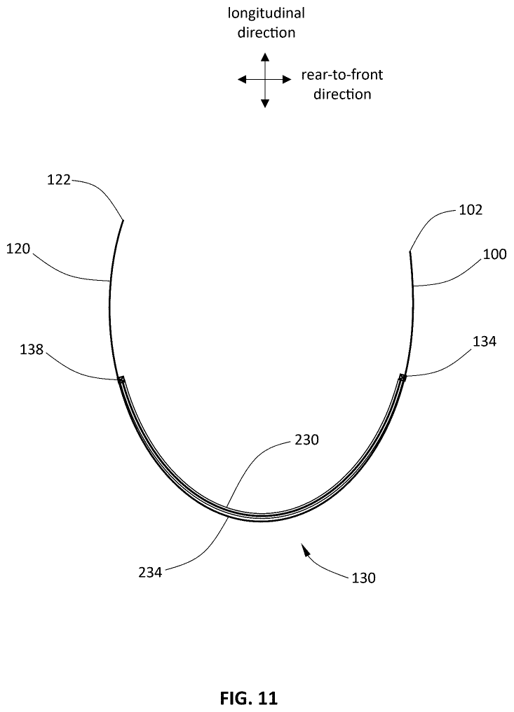

FIG. 9, viewed in conjunction with FIGS. 10A-10C and 11, reflects another approach, referred to herein as a “hammock” configuration. In this configuration, the gusset assembly 230 may be unaffixed to the outward-facing layer 234 of the pant in the crotch portion 130, and/or unaffixed to the outward-facing layer 234 along or proximate the leg edges 104, or even affixed only at forward and rearward ends, along seams 134 and 138 (shown in FIG. 2). Between the seams 134 and 138, the gusset may be unaffixed to the outward-facing layer 234. This de-coupling of the gusset 230 and outward-facing layer 234 allows the outward-facing layer 234 and any elastomeric/elastic components thereof, or affixed thereto, to move and flex independently of the components of the gusset 230, between the forward and rearward seams 134, 138, and provide better ability of the outer pant structure including outward-facing layer 234 and any associated leg band components to urge the gusset assembly against the wearer's body, particularly in configurations in which the gusset assembly might not be as stretchable/elastically extensible as the outer pant structure including outward-facing layer 234. Further, the hammock configuration enables the wearer, if she desires to wear the underpant with a supplemental sanitary napkin of the type having side “wings”, to wrap the wings over the side/leg edges of the gusset and then underneath it, between the gusset and the outward-facing layer 234, thereby concealing the wings behind the outward-facing layer. Depending upon whether the gusset assembly is more or less elastically extensible in a longitudinal direction as the underlying outer pant structure/outward-facing layer 234, it may be desired that the relaxed/contracted length of the gusset assembly between seams 134, 138 be equal to or greater than, or less than, the corresponding relaxed/contracted length of the underlying outward-facing layer(s) 234 between the seam 134, 138 locations. This may be desired to help ensure that the outer pant structure including outward-facing layer 234 is always in longitudinal tension, to help urge the gusset assembly against the wearer's body, through shifting body movements. With respect to FIG. 11, the respective lengths will be the lengths of outward-facing layer 234 and gusset assembly 230, between longitudinally innermost extents of seams 134 and 138 at the longitudinal axis 200 of the pant.

As reflected in FIG. 9, the hammock configuration may include added gusset backing layer 239, disposed beneath the barrier layer. Gusset backing layer 239 may be formed of the same fabric as outward-facing layer 234, or of a differing fabric material. Gusset backing layer 239 may be included to cover, hide and protect barrier layer 233, which in some examples may be fragile and/or may have an undesirably tacky or plastic-like feel and appearance, which the manufacturer may wish to conceal from the wearer's view and touch. In some examples, gusset backing layer 239 may be formed of a fabric that is effectively waterproof (highly resistant to passage of fluid therethrough, under conditions of use contemplated herein—like barrier layer 233 as described above). In such examples, gusset backing layer 239 may supplement or even substitute for and supplant barrier layer 233. Gusset backing layer 239 may be affixed to the absorbent assembly 238 and/or edge sealing strip 235 at the locations shown, by hammock adhesive 239a. Proximate leg edges 104, it may be desired that hammock adhesive 239a be micro-dot adhesive for the reasons explained above, while hammock adhesive 239a may be micro-dot adhesive, or tape or strip adhesive, along forward and rearward gusset seams 134, 138.

FIGS. 10A-10C schematically illustrate longitudinal cross sections of possible examples of configurations for forward and/or rearward seams 134, 138 affixing the gusset assembly 230 to the outward-facing layer 234 in the front 100 and rear 120 portions of the pant.

As reflected in FIG. 10A, a fabric trim strip 237 may be disposed to have one side overlay a forward or rearward end of the gusset 230, and a second side to overlay the outward-facing layer 234 of the pant structure. Trim strip 237 may be affixed at these locations via any suitable mechanism such as adhesive, micro-dot adhesive, stitching, etc. It will be appreciated that concerns for material stacking, binding, stiffening and loss of elasticity may not be as great for forward and rearward seams 134, 138, as for the regions of the pant proximate the leg openings, including leg opening edges 104 in the crotch portion 130.

FIGS. 10B and 10C reflect a more simplified construction, in which material of the gusset backing layer 239 is directly affixed to outward-facing layer 234, at a location that is longitudinally outboard of the edge sealing strip 123 but longitudinally inboard of the folded-over edge of the hammock layer. As reflected in FIG. 10B, the two layers of folded-over gusset backing layer 239 may be stitched with gusset end stitching 241 to the outward-facing layer 234, to form seam 134 (or for a rearward seam, 138). As reflected in FIG. 10c, the outward-facing portion of gusset backing layer 239 may be adhered to the outward-facing layer 234 along seam 134 (or for a rearward seam, 138) by a path of micro-dot gusset end adhesive 242. Noting, however, that concerns for material stacking, binding, stiffening and loss of elasticity may not be as great for forward and rearward seams 134, 138, a tape or strip adhesive may be substituted for micro-dot adhesive 242.

With respect to any of the examples depicted in FIGS. 4A through 6C, 7B, 8, 9, 10A and 10C, it will be noted that the designs reflected do not include stitched seams, but rather, rely upon adhesive, micro-dot adhesive or ultra-sonic bonding to join layers. In some examples of such designs, areas at or proximate the corners of the gusset may be vulnerable to multi-directional stresses that may precipitate separation of the material layers that are adhered or bonded, at the corners. Accordingly, referring back to FIG. 2, it may be desired to include tack stitches 245 proximate to two or all four of the gusset corners, that penetrate and join at least the outward-facing layer 234 and the gusset structure, directly, or indirectly, e.g., via/through a layer overlying wearer-facing layer 231. Such tack stitches, being discrete and localized proximate the gusset corners, will not substantially impair elastic stretch of the materials they join. Where such tack stitches are included, to avoid providing potential leakage pathways from layers to/through which fluids may wick, it may be preferred that the tack stitches do not contact and/or do not penetrate or extend through one or more of absorbent layer(s) 232 and wearer-facing layer 231. For purposes of minimizing their potential impact upon stretch or potential for causing puckering of outward-facing layer 234, such tack stitches, where included, preferably have a largest x-y plane dimension no greater than 15 mm, more preferably no greater than 12 mm, and even more preferably no greater than 10 mm, and still more preferably no greater than 8 mm.

### Other Construction Details

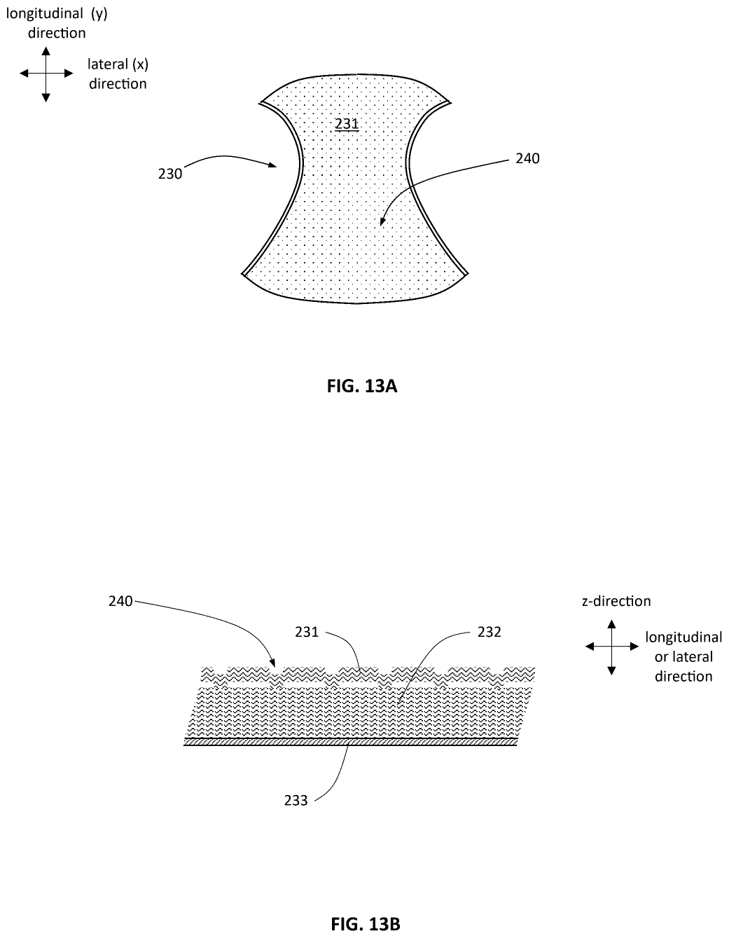

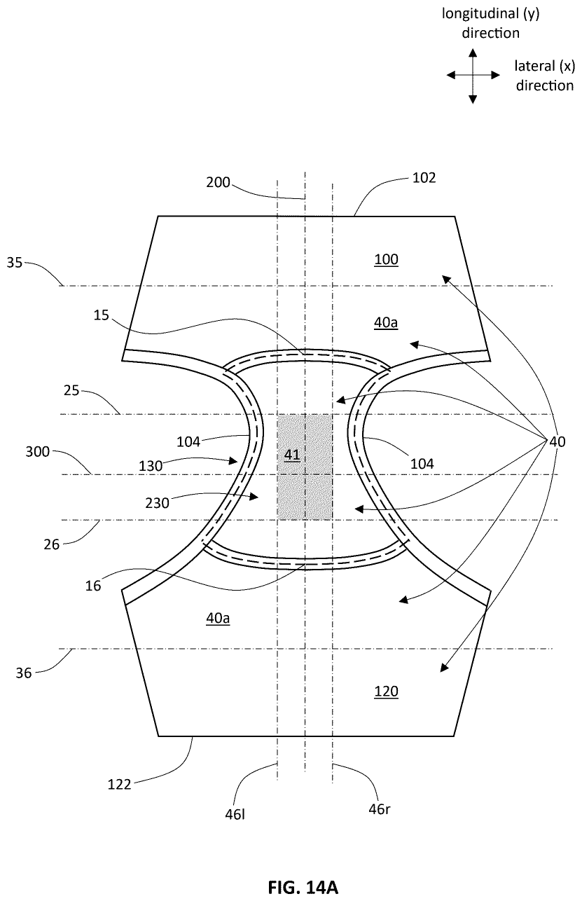

In some examples one or more layers within the crotch portion 130 and/or gusset 230 may be bonded together via any suitable bonding mechanism. Referring to FIGS. 13A and 13B for example, a wearer-facing layer 231 may be bonded to a subjacent absorbent layer 232 for purposes of holding the layers in close proximity and thereby helping maintain a desired low caliper of the structure, and enhancing fluid communication between the layers, i.e., enhancing the structure's ability to pass discharged urine, vaginal, or menstrual fluid from the wearer-facing layer to the underlying absorbent layer, via contact between the layers. In such example, the bonding mechanism should be selected so as not to occlude the interface between the two layers along any substantial portion of their interfacing surface areas. Thus, in some examples it may be desired that the bonding mechanism have the form of a discontinuous pattern of discrete bonds (i.e., a pattern with unbonded regions between the bonds), such as a regular pattern of spot bonds 240. In some examples, bonds between the layers may be formed by discrete deposits of adhesive between the layers, adhering them together at the locations of the deposits. In other examples, bonds between the layers may be formed by thermal compression bonding. The latter bonding mechanism may be more durable through a plurality of launderings, and as illustrated schematically in FIG. 13B, creates a corresponding pattern of z-direction depressions in the wearer-facing layer 231 that may serve to collect discharged urine/fluid and initiate and/or facilitate its movement (via wicking) down to the absorbent layer 232. For purposes of enabling thermal compression bonding, the wearer-facing layer and the subjacent absorbent layer may each include polymer components of respective fusible compositions (such as similar polyester-based compositions) that facilitate formation of robust bonds between the layers upon localized application of heat and pressure at the bond sites. (Herein, “fusible compositions,” with respect to two respective polymers present in two respective layers, means that the two polymers are miscible, capable of melting and mixing at a temperature of 250° C. or lower, to form a single thermodynamic phase.)

Various layers that may be included in crotch portion 130 and/or gusset 230, e.g., layers 231, 232, 233 and 234, may also be joined to each other by any suitable mechanism at forward and rearward seams 134, 138 and crotch side seams 135 proximate the leg edges 104. The joining mechanism may be a system of stitching to affix the layers together; however, for purposes of liquid containment it may be desired that the joining mechanism include a generally hydrophobic, water insoluble adhesive or adhesive film, by itself or as a supplement to stitching, or alternatively, that such stitching be entirely above the barrier layer (not penetrating the barrier layer) or within the containment zone of the edge sealing strip 235.

### Measurement Methods

### General Sample Preparation

Unless otherwise specified below, each of the measurements below is to be conducted on 10 separate like samples (taken from 10 separate like examples of pants) and the average of the 10 separate like samples is considered to be the measurement for that specific sample set.

Referring to FIG. 2, samples including the entire gusset 230 are collected from examples of the subject pant. Lateral lines 234, 238 that are respectively tangent the forwardmost edge of the forward seam 134 and rearward-most edge of the rearward seam 138 are identified, and the pant is cut apart along these lines (without cutting into the seam itself) to provide a sample that includes the entire gusset 230.

If the pant is a legged pant, cut out the gusset in its entirety, along cutting paths outside of the seam(s) joining the gusset to the remainder of the pant, without cutting into the seams themselves. For legged pants, measurements of “Crotch Width” made for purposes herein will be the width of the removed gusset measured along the lateral direction, along a lateral line marking the shortest distance between the leg openings prior to removal of the gusset from the legged pant.

The sample should be cut from the example pant with a sharp knife or suitably sharp cutting device effective to precisely and cleanly cut the sample. A straight edge or other suitable drafting/drawing tool may be used where helpful to hold the example down on the work surface and help guide the cutting device.

The testing is performed under ambient room conditions (temperatures from between 15° C. to 35° C. and relative humidity from between 35% to 75%). Samples are conditioned for at least two hours prior to testing under the same conditions.

All linear dimensions are measured manually by ruler within the ordinary x-y plane, using a ruler that is traceable to NIST or other standards organization.

### Longitudinal Tensile Modulus

For the Longitudinal Tensile Modulus and Maximum Longitudinal Elongation test methods, the samples as described above in “General Sample Preparation” are further modified by cutting out a 40 mm laterally wide section symmetrically about the longitudinal axis 200. The leg edge seams are not included in these tests. If the lateral width between the narrowest separation of leg edge seams as defined by the tangent lines 270 in FIG. 2 is less than 40 mm, then the pant is not deemed to be suitable (as being too narrow through the crotch region for suitable coverage) for purposes described herein, and is considered to be outside the scope of the claims.

The Longitudinal Tensile Modulus of the sample is determined by stretching along the direction of the longitudinal axis 200 of the pant, using a constant rate of extension tensile testing machine with computer interface, e.g., Instron; MTS; Zwick; etc., using a load cell for which the loads measured are within 10% to 90% of the limit of the cell, and ensures accuracy of a 5N load to 0.1N. The instrument is equipped with a single line contact grips, 8 cm in grip width. Prior to testing, calibrate the equipment according to the instruments manufacturer's recommendations.

In accordance with the sample preparation instructions set forth above, the Sample Width is 40 mm. The stress in the sample is calculated by dividing the force in the load cell by the Sample Width, and is expressed in units of gf (grams-force)/mm. (The caliper of the sample is not a factor in this calculation.) The Sample Length is equal to the length of the sample along the longitudinal axis 200 between the lateral lines 234, 238 (along which the sample was cut from the example pant), as illustrated by way of example in FIG. 2.

The grips of the tensile testing machine consist of air actuated grips designed to hold the sample. No slippage should be permitted between the sample and the grips. The distance between the grips (along the axis of the machine's elongation) should be the Sample Length minus 6 cm. This distance will be hereinafter referred to as the “Starting Gauge-Length”.

The sample is mounted in the grips with its longitudinal axis 200 parallel to the direction of applied elongation, and centered in each grip. Two (2) cm of the sample's length at each end is inserted into each grip. The Starting Gauge Length, determined as described above, will ensure that 2 cm of longitudinal slack will be present in the sample at the start of the test.

After the sample is mounted, the machine's load channel is set to zero (this eliminates the weight of the sample in the calculations). The grips are slowly moved apart at 5.08 cm/min (2.0 in/min) until a load of 5 gf (grams-force) is reached. The separation between the grips at this position is recorded as L0.

(L0=Starting Gauge Length+additional machine extension to reach 5 gf)

After the 5 gf load is reached, extend the sample at a rate of 50.8 cm/min (20 in/min) with a data acquisition rate of 50 Hz. Extend until either a stress of 30 gf/mm is reached, or the sample breaks.

Sample strain is calculated by ΔL/L0. ΔL is any additional extension between the grips after L0 is reached and is recorded along with load at a rate of 50 Hz. Sample strain is expressed numerically (not as a percentage), thus a strain of 100% is 1.0 for the purposes of these calculations.

Record the sample strains at sample stresses of 10 gf/mm and at 20 gf/mm.

Longitudinal Tensile Modulus is the linear slope between 10 gf/mm and 20 gf/mm, and is calculated as:

Longitudinal Tensile Modulus=[20 gf/mm−10 gf/mm]/[sample strain at 20 gf/mm−sample strain at 10 gf/mm]

Repeat for 10 samples.

### Maximum Longitudinal Elongation

Maximum Longitudinal Elongation is measured during the Longitudinal Tensile Modulus test. The Maximum Longitudinal Elongation is the sample strain at a sample stress of 20 gf/mm. Maximum Longitudinal Elongation is expressed as a percent strain, e.g., a value of 1.0 strain from the Longitudinal Tensile Modulus method is expressed as 100% strain for Elongation.

Repeat and record the results for 10 samples. Calculate and record the average of the results. The average will be the Maximum Longitudinal Elongation value for the subject pant design.

### Absorption Capacity

The absorption capacity test measures the amount of liquid held within a test sample after specified times of immersion and vertical drainage. The amount of test liquid that is retained by the test sample is used to calculate and report the Area Absorption Capacity (milliliters (ml) of liquid per specimen area in square centimeters) and the Volume Absorption Capacity (in milliliters (ml) of liquid per specimen volume in cubic centimeters). All testing is performed in a room controlled at 23° C.±3° C. and 50%±2% relative humidity.

The test procedure follows compendial method WSP 010.1.R3 (12) part B (Liquid Absorptive Capacity) with modifications specified as follows. The test liquid is deionized water at room temperature (23° C.±3° C.; density 1.00 g/ml). For the weighing portions of the test, no cover glass is used as the test liquid is non-volatile. The overall dimensions of the wire gauze test specimen support is large enough (e.g. 12 inches by 12 inches) to accommodate the larger test sample size. The test sample is the entire gusset (as described herein), thus larger than what is suggested in the compendial method.

Prior to measuring absorption capacity, the examples of the pants of interest (prior to removal of samples therefrom) are washed in order to mimic in-use conditions and to follow the recommended “prior to use” instructions that accompany these types of pants (e.g. wash before use). The examples are placed into a mesh lingerie bag, and then placed into a high efficiency, front-loading washing machine (any convenient source) along with a single small/light load dosage of TIDE brand laundry detergent (“Original” designation; “HE” or other high efficiency washing machine designation; without additives such as FEBREZE, ODOR DEFENSE, OXI additives, bleach or bleaching additives or fabric softening additives) (product of The Procter & Gamble Company, Cincinnati, Ohio). The washer is set to delicate cycle using cold water. After the wash cycle, the examples are removed from the mesh bag and placed flat on a drying rack to air dry for about 12 hours. After air-drying, the examples are placed into a clothes dryer (any convenient source) set on the delicate cycle with very low heat for about 5 minutes or until dry to the touch.

Test samples are prepared as follows. The pre-washed and dried examples are equilibrated in a room controlled at 23° C.±3° C. and 50%±2% relative humidity for about 2 hours. Test samples that include the entire gusset are removed from the examples as described in the General Sample Preparation section herein. Using scissors, the outermost fabric layer on the outward-facing side of the gusset is cut out along the entire gusset shape, inboard of the seams, using care so as not to cut into any of the edge seams present.

The immersion and drainage procedure outlined in the compendial method is then followed with the modifications previously noted. Subtract the Dry Mass from the Wet Mass and record as Liquid Mass Absorbed to the nearest 0.01 grams. Since the density of deionized water is 1.00 g/ml, the Liquid Mass Absorbed is also recorded as Liquid Volume Absorbed to the nearest 0.01 ml. Divide the Liquid Volume Absorbed (ml) by the overall area (cm2) of the test specimen and record as Area Absorption Capacity to the nearest 0.01 ml/cm2. Now divide the Liquid Volume Absorbed (ml) by the volume of the test specimen (area×central caliper) and record as Volume Absorption Capacity to the nearest 0.01 ml/cm3.

In like fashion, repeat for a total of three replicate test specimens. Calculate the arithmetic mean for Area Absorption Capacity and Volume Absorption Capacity and report to the nearest 0.01 ml/cm2 and 0.01 ml/cm3, respectively.

### Caliper

The Caliper of a sample including a gusset is measured as the distance between a reference platform on which the sample rests and a pressure foot that exerts a specified amount of pressure onto the sample over a specified amount of time. All measurements are performed in a laboratory maintained at 23° C.±2° C. and 50%±2% relative humidity.

Caliper is measured with a manually-operated micrometer equipped with a pressure foot capable of exerting a steady pressure of 0.5 kPa±0.01 kPa onto the test sample. The manually-operated micrometer is a dead-weight type instrument with readings accurate to 0.001 mm. A suitable instrument is Mitutoyo Series 543 ID-C Digimatic, available from VWR International, or equivalent. The pressure foot is a flat ground circular movable face with a diameter of 50 mm. The sample is supported by a horizontal flat reference platform that is larger than and parallel to the surface of the pressure foot. The system is calibrated and operated per the manufacturer's instructions.

Obtain a sample including the gusset by removing it from the pant, as described above. When excising the sample from an absorbent article, use care to not impart any wrinkles into the layers or other distortion of the layers during the removal process. Samples are conditioned at 23° C.±2 C° and 50%±2% relative humidity for 2 hours prior to testing. To measure caliper, first zero the micrometer against the horizontal flat reference platform. Place the sample on the platform with the desired measurement location centered below the pressure foot. Gently lower the pressure foot with a descent rate of 1.0 mm±0.1 mm per second until the full pressure is exerted on the sample. Wait 5 seconds and then record the caliper of the sample to the nearest 0.01 mm. In like fashion, repeat for a total of 10 replicate samples. Calculate the arithmetic mean for the Caliper and report to the nearest 0.01 mm.

### Liquid Impermeability

When there is a question concerning whether a particular pant has a gusset with a liquid impermeable barrier layer, this Liquid Impermeability test method may be used to measure a quantity of test liquid that will pass through a sample and enable determination whether there is a “liquid impermeable barrier layer” present in the gusset, according to the definition set forth in the description above.

The Liquid Impermeability test measures the quantity of liquid transferred through to the outward-facing side of a test specimen obtained from a pant after it is dosed with a prescribed volume of test liquid in order to simulate a liquid insult during actual use/wear of the pant.

All testing is performed in a room controlled at 23° C.±3° C. and 50%±2% relative humidity.

Prior to performing the measurement of this method, the examples of the pants of interest (prior to removal of samples therefrom) are washed in order to mimic in-use conditions and to follow the recommended “prior to use” instructions that accompany these types of pants (e.g. wash before use). The examples are placed into a mesh lingerie bag, and then placed into a high efficiency, front-loading washing machine (any convenient source) along with a single small/light load dosage of TIDE brand laundry detergent (“Original” designation; “HE” or other high efficiency washing machine designation; without additives such as FEBREZE, ODOR DEFENSE, OXI additives, bleach or bleaching additives or fabric softening additives) (product of The Procter & Gamble Company, Cincinnati, Ohio), or equivalent. The washer is set to delicate cycle using cold water. After the wash cycle, the examples are removed from the mesh bag and placed flat on a drying rack to air dry for about 12 hours. After air-drying, the examples are placed into a clothes dryer (any convenient source) set on the delicate cycle with very low heat for about 5 minutes or until dry to the touch.

Test samples are prepared as follows. The pre-washed and dried example pants are equilibrated in a room controlled at 23° C.±3° C. and 50%±2% relative humidity for about 2 hours. Test samples containing the entire gusset are removed from the examples as described in the General Sample Preparation section herein. Using scissors, the outermost fabric layer on the outward-facing side of the gusset is cut out along the entire gusset shape, inboard of the seams, using care so as not to cut into any of the edge seams present. Mark the dose location at the intersection of the midpoint of the longitudinal axis of the sample and a lateral axis positioned at the narrowest portion of the specimen.

For each test sample, a single layer of filter paper is cut to 10 cm by 2.54 cm. A suitable filter paper is Ahlstrom Grade 989 (available from Ahlstrom-Monks North America LLC, Alpharetta, GA), or equivalent. The test liquid is deionized water at room temperature (23° C.±3° C.).

Record the mass of one layer of pre-cut filter paper and record as Dry Massfp to the nearest 0.0001 grams. Place the pre-weighed filter paper onto a flat horizontal work surface. Position the test specimen centered over the filter paper with the garment facing side of the specimen facing the paper. Using a volumetric pipette, apply a 1.0 ml dose of test liquid to the pre-marked dosing location as follows. The tip of the pipette is held about 3 mm above the surface of the test specimen, and the dose is applied slowly (about 30 seconds) to avoid splashing. As soon as the entire dose has been applied, start a 1 minute timer. After 1 minute has elapsed, remove the test specimen and record the mass of the filter paper as Wet Massfp to the nearest 0.0001 grams. Subtract the Dry Massfp from the Wet Massfp and record as z-Direction Leakage to the nearest 0.0001 grams.

In like fashion, repeat for a total of three replicate test specimens. Calculate the arithmetic mean for z-Direction Leakage and report to the nearest 0.0001 g.

In view of the foregoing disclosure, the following non-limiting examples are contemplated:

1. A durable absorbent pant having a front waist portion, a rear waist portion and a crotch portion; the front portion and rear portions meeting each other at side hip areas, and meeting the crotch portion, the front, rear and crotch portions defining a waist opening edge and a pair of leg opening edges; the pant comprising:
an absorbent gusset disposed in at least the crotch portion, the absorbent gusset comprising at least one layer of absorbent fabric and a fluid barrier layer disposed beneath the at least one layer of absorbent fabric; the absorbent fabric having a forwardmost end edge in the front portion, a rearwardmost end edge in the rear portion, and a pair of opposing side edges proximate the respective leg opening edges;
a peripheral region; and
an odor-adsorbent structure disposed at least partially in the peripheral region, the odor-adsorbent structure comprising an adsorbent layer comprising an adsorbent material selected from the group consisting of zeolites, functionalized silicas metal-organic frameworks, activated carbon, and combinations thereof.
   wherein a basis weight of adsorbent material present is greater in the peripheral region than in a central region.
   2. The durable absorbent pant of example 1 wherein the odor adsorbent structure comprises an overlying protective layer comprising vapor-permeable fabric, whereby vapor and gas may penetrate the vapor-permeable fabric and contact the adsorbent material.
   3. The durable absorbent pant of example 2 wherein the protective layer is a wearer-contacting layer.
   4. The durable absorbent pant of either of examples 2 or 3 wherein the protective layer comprises knit, woven, or film materials comprising components selected from the group consisting of polyolefin, polyester, polyamide, and combinations thereof, and has a basis weight of 20 gsm to 80 gsm and a porous woven, knitted and/or apertured structure.
   5. The durable absorbent pant of any of the preceding examples wherein a basis weight of adsorbent material present is greatest in a near peripheral region 40a.
   6. The durable absorbent pant of any of the preceding examples wherein the central region 41 contains less than 50 weight percent, preferably less than 25 percent, more preferably less than 10 percent, and even more preferably less than 5 percent, of the odor-adsorbent material present.
   7. The durable absorbent pant of any of the preceding examples wherein the odor-adsorbent structure is located entirely in the peripheral region 40.
   8. The durable absorbent pant of any of the preceding examples wherein the odor-absorbent structure is located entirely in the near peripheral region 40a.
   9. The durable absorbent pant of any of the preceding examples wherein some or all of the adsorbent material is in the form of particulates or granules.
   10. The durable absorbent pant of example 9 wherein the particulates or granules are affixed to a substrate with a binder.
   11. The durable absorbent pant of any of examples 1-8 wherein the adsorbent material comprises activated carbon.
   12. The durable absorbent pant of example 11 wherein some or all of the activated carbon is in the form of fibers.
   13. The durable absorbent pant of example 12 wherein the fibers are components of a fabric.
   14. The durable absorbent pant of any of the preceding examples wherein the protective layer comprises fiber, film, and/or yarn components with hydrophobic surfaces.
   15. The durable absorbent pant of any of the preceding examples wherein the odor adsorbent structure is elongate, with a longer dimension that is oriented predominantly laterally, and the structure is disposed in front and/or rear portions of the peripheral region, and preferably both front and rear portions of the peripheral region.
   16. The durable absorbent pant of any of the preceding examples wherein the odor adsorbent structure comprises a substrate layer (58) underlying the adsorbent layer.
   17. The durable absorbent pant of any of the preceding examples wherein the odor adsorbent structure is affixed to a wearer-facing surface of the front waist portion, rear waist portion or crotch portion by an adhesive material.
   18. The durable absorbent pant of any of the preceding examples wherein the odor adsorbent structure comprises, by weight, less than 0.1 percent, preferably less than 0.05 percent, metals or metallic compounds, and more preferably, is substantially free of metals or metallic compounds.
   19. The durable absorbent pant of any of the preceding examples wherein a layer of the odor adsorbent structure (55), that is visible among wearer-facing surfaces of the underwear, has a color that visibly contrasts with one or materials of the underwear adjacent the odor adsorbent structure.
   20. The durable absorbent pant of any of the preceding examples wherein the odor adsorbent layer(s) present have a combined x-y plane surface area totaling 2.5 percent to 400 percent of the total x-y plane surface area of the wearer-facing surface(s) of all absorbent layer(s) of the gusset, including all wearer-facing surface areas of all absorbent layers present.
   21. The durable absorbent pant of any of the preceding examples wherein the odor adsorbent structure has a caliper not exceeding 5 mm.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A durable absorbent pant having a front waist portion, a rear waist portion and a crotch portion; the front portion and rear portions meeting each other at side hip areas, and meeting the crotch portion, the front, rear and crotch portions defining a waist opening edge and a pair of leg opening edges; the pant comprising:
an absorbent gusset disposed in at least the crotch portion, the absorbent gusset comprising at least one layer of absorbent fabric and a fluid barrier layer disposed beneath the at least one layer of absorbent fabric; the absorbent fabric having a forwardmost end edge in the front portion, a rearwardmost end edge in the rear portion, and a pair of opposing side edges proximate the respective leg opening edges;
a peripheral region; and
an odor-adsorbent structure disposed at least partially in the peripheral region, the odor-adsorbent structure comprising an adsorbent layer comprising an adsorbent material selected from the group consisting of zeolites, functionalized silicas metal-organic frameworks, activated carbon, and combinations thereof,
wherein a basis weight of adsorbent material present is greater in the peripheral region than in a central region.

2. The durable absorbent pant of claim 1 wherein the odor adsorbent structure comprises an overlying protective layer comprising vapor-permeable fabric, whereby vapor and gas may penetrate the vapor-permeable fabric and contact the adsorbent material.

3. The absorbent pant of claim 2 wherein the protective layer is a wearer-contacting layer.

4. The absorbent pant of claim 2, wherein the protective layer comprises knit, woven, or film materials comprising components selected from the group consisting of polyolefin, polyester, polyamide, and combinations thereof, and has a basis weight of 20 gsm to 80 gsm and a porous woven, knitted and/or apertured structure.

5. The absorbent pant of claim 1, wherein a basis weight of adsorbent material present is greatest in a near peripheral region.

6. The absorbent pant of claim 1, wherein the central region contains less than 50 weight percent of the odor-adsorbent material present.

7. The absorbent pant of claim 1, wherein the odor-adsorbent structure is located entirely in the peripheral region.

8. The absorbent pant of claim 1, wherein the odor-absorbent structure is located entirely in the near peripheral region.

9. The absorbent pant of claim 1, wherein some or all of the adsorbent material is in the form of particulates or granules.

10. The absorbent pant of claim 9, wherein the particulates or granules are affixed to a substrate with a binder.

11. The absorbent pant of claim 1, wherein the adsorbent material comprises activated carbon.

12. The absorbent pant of claim 11, wherein some or all of the activated carbon is in the form of fibers.

13. The absorbent pant of claim 12, wherein the fibers are components of a fabric.

14. The absorbent pant of claim 1, wherein the protective layer comprises fiber, film, and/or yarn components with hydrophobic surfaces.

15. The absorbent pant of claim 1, wherein the odor adsorbent structure is elongate, with a longer dimension that is oriented predominantly laterally, and the structure is disposed in front and/or rear portions of the peripheral region, and preferably both front and rear portions of the peripheral region.

16. The absorbent pant of claim 1, wherein the odor adsorbent structure comprises a substrate layer underlying the adsorbent layer.

17. The absorbent pant of claim 1, wherein the odor adsorbent structure is affixed to a wearer-facing surface of the front waist portion, rear waist portion or crotch portion by an adhesive material.

18. The absorbent pant of claim 1, wherein the odor adsorbent structure comprises, by weight, less than 0.1 percent metals or metallic compounds.

19. The absorbent pant of claim 1, wherein the odor adsorbent layer(s) present have a combined x-y plane surface area totaling 2.5 percent to 400 percent of the total x-y plane surface area of the wearer-facing surface(s) of all absorbent layer(s) of the gusset, including all wearer-facing surface areas of all absorbent layers present.

20. The absorbent pant of claim 1, wherein the odor adsorbent structure has a caliper not exceeding 5 mm.
